(19) United States
(12) Patent Application Publication
(10) Pub. No.: US 2009/0192077 A1
(43) Pub. Date: Jul. 30, 2009

(54) North-2'deoxy -methanocarbathymidines as antiviral agents for treatment of kaposi's sarcoma-associated herpes virus

(76) Inventors: Shizuko Sei, Bethesda, MD (US); Victor Marquez, Montgomery Village, MD (US); Robert H. Shoemaker, Boyds, MD (US)

Correspondence Address: KNOBBE, MARTENS, OLSON & BEAR, LLP, 2040 MAIN STREET, FOURTEENTH FLOOR, IRVINE, CA, 92614, US

(21) Appl. No.: 11/918,510

(22) PCT Filed: Apr. 11, 2006
(86) PCT No.: PCT/US2006/013272

§ 371 Date: Dec. 30, 2008

Related U.S. Application Data
(60) Provisional application No. 60/671,691, filed on Apr. 15, 2005.

(51) Int. Cl. A61K 38/14; A61K 31/513; A61K 31/7048; A61K 31/704; A61P 31/12

(52) U.S. Cl. 514/8; 514/274; 514/27; 514/34

(57) ABSTRACT

A method for the prevention or treatment of Kaposi's sarcoma or Kaposi's sarcoma-associated herpes virus infection by administering an effective amount of a cyclopropanated carbocyclic 2′-deoxynucleoside to an individual in need thereof is provided.

### RELATED APPLICATIONS

This application claims the benefit of priority of U.S. Provisional application No. 60/671,691, filed Apr. 15, 2005, the disclosure of which is hereby expressly incorporated by reference in its entirety.

### FIELD OF THE INVENTION

A method for the prevention or treatment of Kaposi's sarcoma or Kaposi's sarcoma-associated herpes virus infection by administering an effective amount of a cyclopropanated carbocyclic 2′-deoxynucleoside to an individual in need thereof is provided.

### BACKGROUND OF THE INVENTION

Kaposi's sarcoma (KS) is a multifocal malignant tumor of endothelial cell origin characterized by the proliferation of spindle-shaped cells with aberrant neovascularization and a large inflammatory cell infiltrate (Boshoff, C. et al. 2001 Philos Trans R Soc Lond B Biol Sci 356:517-34; Ensoli, B. et al. 2001 Eur J Cancer 37:1251-69). KS usually manifests as pigmented nodular skin lesions, but can often spread to visceral organs in immunocompromised hosts, including patients with AIDS (Friedman-Kien, A. et al. 1990 J Am Acad Dermatol 22:1237-50; Lemlich, G., et al. 1987 J Am Acad Dermatol 16:319-25) and organ-transplant recipients (Farge, D. 1993 Eur J Med 2:339-43; Qunibi, W. et al. 1988 Am J Med 84:225-32; Shepherd, F. et al. 1997 J Clin Oncol 15:2371-7). This aggressive and disseminated form of KS was recognized as one of the first AIDS-defining conditions at the beginning of the HIV epidemic in the early 1980s (MMWR Morb Mortal Wkly Rep 30:305-8; Gottlieb, M. et al. 1981 N Engl J Med 305:1425-31; Hymes, K. et al. 1981 Lancet 2:598-600; Masur, H. et al. 1981 N Engl J Med 305:1431-8; Siegal, F. et al. 1981 N Engl J Med 305:1439-44). Without effective therapy, visceral KS can be highly fatal, unless the underlying causes of immune suppression are successfully treated (Aboulafia, D. M. 1998 Mayo Clin Proc 73:439-43; Dupont, C. et al. 2000 AIDS 14:987-93; Gill, J. et al. 2002 J Acquir Immune Defic Syndr 31:384-90; Tirelli, U. et al. 2001 Eur J Cancer 37:1320-4). Cytotoxic chemotherapeutic agents are commonly used in disseminated KS with response rates of up to 80% (Evans, S. R. et al. 2002 J Clin Oncol 20:3236-41; Gill, P. et al. 1990 Am J Clin Oncol 13:315-9; Gill, P. S. et al. 1991 Am J Med 90:427-33; Newell, M. et al. 1998 Aust N Z J Med 28:777-83; Welles, L. et al. 1998 J Clin Oncol 16:1112-21). However, the majority of these agents are associated with serious side effects, and the tumor response to any chemotherapeutic regimen is only transient. There is no definitive cure for KS at the present time.

KS-associated herpesvirus (KSHV, also called human herpesvirus 8 or HHV8) was first discovered in KS lesions obtained from AIDS patients (Chang, Y. et al. 1994 Science 266:1865-9; Foreman, K. E. et al. 1997 N Engl J Med 336:163-71). It was subsequently found in all forms of KS and has strongly been implicated in the pathogenesis of KS (Chang, Y. et al. 1996 Arch Intern Med 156:202-4; Moore, P. S. et al. 1995 N Engl J Med 332:1181-5). KSHV is a γ2-herpesvirus (genus Rhadinovirus) closely related to other oncogenic γ-herpesviruses, including herpesvirus saimiri (γ2), murine gammaherpesvirus (γ2) and Epstein-Barr virus (EBV) (γ1) (Moore, P. S. et al. 1996 J Virol 70:549-58). Since its discovery, KSHV has also been linked to a rare form of AIDS-associated effusion-based B cell lymphoma, termed primary effusion lymphoma or body cavity based lymphoma (BCBL) (Cesarman, E. et al. 1995 N Engl J Med 332:1186-91), and a subset of multicentric Castleman's disease (Soulier, J. et al. 1995 Blood 86:1276-80). Although the exact etiologic mechanism of these neoplastic disorders is still unclear, KSHV infection is believed to play a critical role in the tumorigenesis and/or tumor progression. A number of studies have shown that higher levels of KSHV viral load in peripheral blood mononuclear cells or serum antibody titers against KSHV proteins correlated with increased risk of KS in HIV-infected (Engels, E. A. et al. 2003 AIDS 17:1847-51; Renwick, N. et al. 1998 AIDS 12:2481-8; Rezza, G. et al. 1999 J Natl Cancer Inst 91:1468-74; Whitby, D. et al. 1995 Lancet 346:799-802) and uninfected individuals (Farge, D. et al. 1999 Transplantation 67:1236-42; Pellet, C. et al. 2002 J Infect Dis 186:110-3). Higher KSHV viral load in peripheral blood has also been associated with progressive KS in HIV-infected individuals (Campbell, T. B. et al. 2000 AIDS 14:2109-16; Lallemand, F. et al. 2000 J Clin Microbiol 38:1404-8; Quinlivan, E. B. et al. 2002 J Infect Dis 185:1736-44). Moreover, several clinical studies, including one prospective study, have found that the risk of KS was lower in AIDS patients, who received ganciclovir (GCV) or foscarnet for cytomegalovirus (CMV) infection (Glesby, M. J. et al. 1996 J Infect Dis 173:1477-80; Martin, D. F. et al. 1999 N Engl J Med 340:1063-70; Mocroft, A. et al. 1996 AIDS 10:1101-5), suggesting that the use of anti-herpesvirus agents may have deterred the development of KS, presumably by inhibiting KSHV lytic replication.

To exploit the involvement of KSHV in the tumorigenesis, KSHV-targeted molecular intervention has been proposed to treat KS and other KSHV-induced malignancies, including the use of GCV and foscarnet as anti-herpetic DNA synthesis inhibitors (Krown, S. E. 2003 Hematol Oncol Clin North Am 17:763-83).

Nucleoside analogs lacking 2′- and 3′-hydroxyl groups (dideoxynucleosides), as well as those 2′-deoxynucleosides where the 3′-hydroxyl function has been chemically modified or changed, can function as chain terminators of DNA synthesis after their triphosphate metabolites are incorporated into DNA. This is the basis of the Sanger dideoxynucleotide method for DNA sequencing (Sanger et al. 1977 PNAS USA 74:5463-5467). Intense effort has focused on the design and use of these compounds as inhibitors of viral replication (Van Roey et al. 1990 Ann NY Acad Sci 616:29). Although the conformation of the sugar moiety in these analogs is believed to play a critical role in modulating biological activity, including the anti-HIV activity mediated by derivatives such as azidothymidine (AZT) and dideoxyinosine (ddI), the main problem encountered in correlating a specific type of sugar conformation with the biological activity of nucleoside analogs is that the sugar ring is quite flexible and its conformation in solution can differ markedly from its conformation in the solid state (Jagannadh et al. 1991 Biochem Biophys Res Commun 179:386-391; Plavec et al. 1992 Biochem Biophys Methods 25:25-272). Thus, for nucleosides in general, any structure-activity analysis which is based solely on the solid-state conformation would be inaccurate unless it was previously determined that both solution and solid-state conformations were the same.

In solution there is a dynamic equilibrium between Northern (N) and Southern (S) type furanose conformers (Taylor et al. 1990 Antiviral Chem Chemother 1:163-173) as defined in the pseudorotational cycle. In this cycle, an absolute Northern conformation corresponds to a range of P (angle of pseudorotation) of from 342° to 18° (₂E→³T₂→³E), whereas an absolute Southern conformation corresponds to a range of P of from 162° to 198° (²E→²T₃→₃E). Preference for any of these specific conformations in solution is determined by the interplay of interactions resulting from anomeric and gauche effects (Saenger, in Principles of Nucleic Acid Structure, Springer-Verlag, New York, pp. 51-104, 1984; Plavec et al. 1972 J Am Chem Soc 94:8205-8212). When a nucleoside or nucleotide binds to its target enzyme, only one form is expected to be present at the active site. While the energy gap between Northern and Southern conformations is about 4 kcal/mol, such a disparity can explain the difference between micromolar and nanomolar binding affinities.

The conformations of nucleosides and their analogs can be described by the geometry of the glycosyl link (syn or anti), the rotation about the exocyclic C4′-C5′ bond and the puckering of the sugar ring leading to formation of the twist and envelope conformations. Two types of sugar puckering are generally energetically preferred, namely the C2′-exo/C3′-endo (N or Northern) and the C2′-endo/C3′-exo (S or Southern). The terms “endo” and “exo” refer to displacement of the atom above or below the plane of the ribose ring, respectively. The torsion angles χ [C2-N1-C1′-O4′ (pyrimidines) or C4-N9-C1′-O4′ (purines)] and γ(C3′-C4′-C5′-O5′) describe, respectively, the orientations of the base and the 5′-hydroxyl group relative to the ribose ring.

In DNA duplexes, a Southern conformation of the repeating nucleoside unit confers upon the double helix a B-conformation, whereas the Northern conformation induces an A-conformation double helix. The A and B forms of DNA differ in the number of base pairs per turn, the amount of rotation per base pair, the vertical rise per base pair and the helical diameter. In addition, in stretches of DNA containing alternating purines and pyrimidines, a left-handed helix called Z-DNA may form.

Altmann et al. demonstrated that substitution of N-methanocarba-thymidine ((N)-methanocarba-T) for thymidine in DNA/RNA heteroduplexes increased the thermodynamic stability of the double helix, as indicated by a positive increase in the Tₘ, whereas the Southern conformer induced a small destabilizing effect (Altmann et al., Tetrahedron Lett., 35:7625-7628, 1994). The increased thermal stability reported for two different (N)-methanocarba-T-containing oligodeoxynucleotides (ODNs) versus conventional ODNs was between 0.8 and 2.1° for a single modified nucleotide; however, no data was reported for an ODN containing multiple (N)-methanocarba-Ts in this study. To further elucidate the stabilizing effect of multiple (N)-methanocarba-Ts in the context of the DNA/RNA heteroduplex, a test sequence targeted to the coding region of the SV40 large T-antigen (Wagner, R. W. et al. 1993 Science 260:1510-13) was subsequently synthesized as the phosphorothioate 5′-CTTCATTTTTTCTTC-3′ (SEQ ID NO: 1), where all thymidines (T) were replaced by (N)-methanocarba-Ts (Marquez, V. E. et al. 1996 J Med Chem 39:3739-47). The additive increase in thermodynamic stability of the heteroduplex due to the presence of multiple (N)-methanocarba-T nucleotides was clearly demonstrated with the average stabilization per substitution of ca. 1.3° C. relative to thymidine (Marquez, V. E. et al. 1996 J Med Chem 39:3739-47).

Conformationally (Northern) locked nucleoside analogs are described in U.S. Pat. No. 5,629,454 and in U.S. Pat. No. 5,869,666.

### SUMMARY OF THE INVENTION

There is a need for effective anti-KS and anti-KSHV agents. The compositions and methods of the preferred embodiments provide such agents and associated methods of treatment.

North-methanocarbathymidine (N-MCT), a thymidine analog with a pseudosugar moiety locked in the northern conformation, which was previously shown to exert strong antiviral activity against herpes simplex virus types 1 and 2 (Marquez, V. E. et al. 1996 J Med Chem 39:3739-47), has been identified as exhibiting potent anti-ICS and anti-KSHV activity. N-MCT effectively blocks KSHV DNA synthesis through its triphosphate (TP) metabolite, which is more efficiently produced in KSHV infected cells. N-MCT is 5- to 10-fold more active than previously identified inhibitors of KSHV DNA synthesis, cidofovir (CDV) and GCV. The higher potency and target specificity of N-MCT against KSHV makes it a more desirable anti-KS and anti-KSHV agent.

In a first aspect, a method of treating a Kaposi's sarcoma-associated herpes virus infection in an individual in need thereof is provided, the method comprising the step of administering to the individual an effective Kaposi's sarcoma-associated herpes virus antiviral amount of a compound having the formula

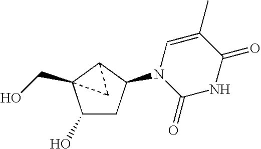

or a triphosphate thereof, in a pharmaceutically acceptable carrier.

In an embodiment of the first aspect, the effective Kaposi's sarcoma-associated herpes virus antiviral amount is from about 300 mg per day to about 15,000 mg per day.

In an embodiment of the first aspect, the step of administering is selected from the group consisting of topical administration, oral administration, intraocular administration intravenous administration, intramuscular administration, parenteral administration, intradermal administration, intraperitoneal administration, and subcutaneous administration.

In a second aspect, a method of treating a Kaposi's sarcoma-associated herpes virus infection in an individual in need thereof is provided, comprising the step of administering to the individual an effective Kaposi's sarcoma-associated herpes virus antiviral amount of North-methanocarbathymidine triphosphate.

In a third aspect, a pharmaceutical kit is provided comprising an antiviral agent comprising a compound having the formula

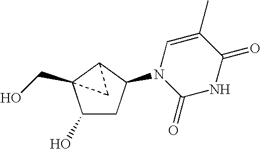

or a triphosphate thereof, in a pharmaceutically acceptable carrier; and directions for administering the antiviral agent to a patient in need thereof for treatment of a Kaposi's sarcoma-associated herpes virus infection.

In an embodiment of the third aspect, the kit further comprises a reverse transcriptase inhibitor selected from the group consisting of zidovudine, didanosine, zalcitabine, stavudine, 3TC, and nevirapine

In an embodiment of the third aspect, the kit further comprises a protease inhibitor and directions for administering the protease inhibitor to the patient.

In an embodiment of the third aspect, the kit further comprises a cytokine and directions for administering the cytokine to the patient.

In an embodiment of the third aspect, the kit further comprises an immunomodulator and directions for administering the immunomodulator to the patient.

In a fourth aspect, a method of treating a Kaposi's sarcoma in an individual in need thereof is provided, comprising the step of administering to the individual an effective amount of a compound having the formula

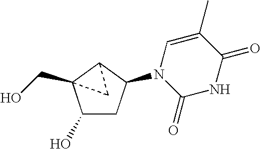

or a triphosphate thereof, in a pharmaceutically acceptable carrier.

In an aspect of the fourth embodiment, the effective amount is from about 40 mg per day to about 15,000 mg per day.

In an aspect of the fourth embodiment, the step of administering is selected from the group consisting of topically administering, orally administering, intravenously administering, intramuscularly administering, parenterally administering, intradermally administering, intraperitoneally administering, and subcutaneously administering

In a fifth aspect, a method of treating a Kaposi's sarcoma in an individual in need thereof is provided, comprising the step of administering to the individual an effective amount of North-methanocarbathymidine triphosphate.

In a sixth aspect, a pharmaceutical kit is provided comprising an anticancer agent comprising a compound having the formula

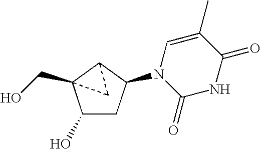

or a triphosphate thereof, in a pharmaceutically acceptable carrier; and directions for administering the anticancer agent to a patient in need thereof for treatment of a Kaposi's sarcoma.

In an embodiment of the sixth aspect, the kit further comprises a chemotherapeutic agent selected from the group consisting of topoisomerase II inhibitors, antibiotics, vinca alkaloids, anthracyclines, and taxanes; and directions for administering the chemotherapeutic agent to the patient.

In an embodiment of the sixth aspect, the topoisomerase II inhibitor comprises etoposide.

In an embodiment of the sixth aspect, the antibiotic comprises bleomycin.

In an embodiment of the sixth aspect, the vinca alkaloid comprises vincristine or vinblastine.

In an embodiment of the sixth aspect, the anthracycline comprises doxorubicin or daunorubicin.

In an embodiment of the sixth aspect, the taxane comprises paclitaxol.

In an embodiment of the sixth aspect, the kit further comprises an angiogenesis inhibitor and directions for administering the angiogenesis inhibitor to the patient.

In an embodiment of the sixth aspect, the angiogenesis inhibitor is selected from the group consisting of thalidomide, angiostatin, semaxinib, and endostatin.

In an embodiment of the sixth aspect, the kit further comprises interferon-alpha and directions for administering the interferon-alpha to the patient.

In an embodiment of the sixth aspect, the kit further comprises alitretinoin and directions for administering the alitretinoin to the patient.

In an aspect of the sixth embodiment, the kit comprises a chemotherapeutic agent selected from the group consisting of etoposide, bleomycin, vincristine, vinblastine, doxorubicin, daunorubicin, and paclitaxol; and directions for administering the chemotherapeutic agent to the patient.

In an aspect of the sixth embodiment, the kit comprises an angiogenesis inhibitor and directions for administering the angiogenesis inhibitor to the patient.

In an aspect of the sixth embodiment, the angiogenesis inhibitor is selected from the group consisting of thalidomide, angiostatin, semaxinib, and endostatin.

In an aspect of the sixth embodiment, the kit comprises interferon-alpha and directions for administering the interferon-alpha to the patient.

In an aspect of the sixth embodiment, the kit comprises alitretinoin and directions for administering the alitretinoin to the patient.

### BRIEF DESCRIPTION OF THE DRAWINGS

FIGS. 1A and 1B depict the effects of N-MCT, cidofovir (CDV) and ganciclovir (GCV) on KSHV DNA replication (FIG. 1A) and cell growth (FIG. 1B).

FIG. 2 provides intracellular phosphorylation profiles of N-MCT in KSHV-infected BCBL-1 cells (FIG. 2A) and uninfected CEM-SS cells (FIG. 2B), with and without PMA (phorbol-12-myristate-13-acetate) stimulation. PMA-stimulated (solid lines) and unstimulated cells (dotted lines) were incubated with 10 μM N-MCT and 5 μCi/mL of [³H]-(N)-MCT for 6, 24 or 72 hours. Methanolic extracts obtained from the harvest cells were subjected to HPLC separation of mono-, di- and tri-phosphorylated metabolites, N-MCT-MP, -DP and -TP, respectively. The data shown are representative of two independent experiments.

FIG. 3 provides intracellular phosphorylation profiles of N-MCT, CDV, and GCV in BCBL-1 cells with and without PMA stimulation. PMA-stimulated (+PMA) and unstimulated BCBL-1 cells (no PMA) were incubated with 10 μM N-MCT, CDV or GCV and 5 μCi/mL of [³H]-(N)-MCT, [³H]-CDV or [³H]-GCV for 24 (top) or 72 hours (bottom). Methanolic extracts obtained from the harvested cells were analyzed for the mono- (MP), di- (DP) and tri-phosphorylated metabolites (-TP). Of note, the phosphorylated metabolites of CDV were identified as CDV-phosphate (CDV-MP), CDV-DP (active metabolite) and a phosphate ester adduct of CDV (CDV-adduct) as previously described (Ho, H. et al. 1992 Mol Pharmacol 41:197-202). The data shown are mean±SD of two separate assays.

FIG. 4A depicts the effects of a potent inhibitor of HSV-1 TK, 5′-ethynylthymidine (5′-ET) (Nutter, L. M. et al. 1987, Antimicrob Agents Chemother 31:368-74) on the anti-KSHV activity of N-MCT or CDV in PMA-stimulated (PMA+) BCBL-1 cells. The levels of cytoplasmic KSHV DNA evaluated by ORF65 PCR were markedly increased in the cells treated with a combination of 1 μM N-MCT and 10, 20 or 50 μM 5′-ET, as compared to the cells treated with 1 μM N-MCT alone, whereas there was no notable difference between cells treated with 10 μM CDV alone or in combination with 5′-ET.

FIG. 4B depicts the effects of 5′-ET (50 μM) on anti-KSHV activity of N-MCT used at 1, 3, or 10 μM. The amounts of virion-associated (supernatants) and cytoplasmic KSHV DNA (LMW) determined by ORF65 PCR were significantly higher in the cells treated with both N-MCT and 5′-ET than the cells treated with N-MCT alone at all three concentrations.

FIG. 4C depicts the levels of phosphorylated metabolites of N-MCT added at 1, 3, or 10 μM in the absence (top) or presence (bottom) of 50 μM 5′-ET in PMA-stimulated BCBL-1 cells. A dose-dependent increase in the intracellular levels of N-MCT-MP, -DP and -TP were observed in the cells treated with 1, 3, or 10 μM N-MCT alone (top). In the presence of 5′ET, the levels of N-MCT-DP and N-MCT-TP were substantially decreased, while N-MCT-MP levels appear to increase (bottom).

FIG. 5 depicts the inhibitory activity of N-MCT-TP (N-MCT triphosphate), CDV-DP (cidofovir diphosphate), or GCV-TP (ganciclovir triphosphate) on in vitro DNA synthesis mediated by recombinant KSHV polymerase (rPOL) and polymerase processivity factor (rPPF). All three phosphorylated compounds dose-dependently blocked KSHV rPOL/rPPF-mediated DNA synthesis with the order of potency N-MCT-TP, CDV-DP and GCV-TP shown as % inhibition (mean±SD of triplicate wells). N-MCT-TP was the only compound that achieved greater than 90% inhibition within the concentrations tested (up to 500 μM). The inhibitory activity of CDV-DP appeared to level off around 60 to 70%. The results shown are representative of three independent experiments.

### DETAILED DESCRIPTION OF THE PREFERRED EMBODIMENT

The following description and examples illustrate some exemplary embodiments of the disclosed invention in detail. Those of skill in the art will recognize that there are numerous variations and modifications of this invention that are encompassed by its scope. Accordingly, the description of a certain exemplary embodiment should not be deemed to limit the scope of the present invention.

Kaposi's sarcoma-associated herpesvirus (KSHV) infection is a prerequisite for the development of Kaposi's sarcoma (KS). Blocking lytic KSHV replication may hinder KS tumorigenesis. North-methanocarbathymidine (N-MCT), a thymidine analog with a pseudosugar ring locked in the northern conformation, exhibits exceptionally potent in vitro anti-KS and anti-KSHV activity. N-MCT inhibits KSHV virion production without cytotoxicity in KSHV-infected BCBL-1 cells lytically-induced by phorbol ester (PMA) with a substantially lower 50% inhibitory concentration (IC₅₀) than those of cidofovir (CDV) and ganciclovir (GCV) (IC₅₀, mean±SD: 0.08±0.03, 0.42±0.07 and 0.96±0.49 μM for N-MCT, CDV and GCV, respectively). The inhibition of KSHV virion production coincides with a dose-dependent decrease in cytoplasmic KSHV DNA levels, indicating that N-MCT blocked lytic KSHV DNA replication. A time and dose-dependent accumulation of N-MCT-triphosphate (TP) was demonstrated in PMA-stimulated BCBL-1 cells, while uninfected cells showed virtually no accumulation regardless of PMA stimulation. The levels of N-MCT-TP were significantly decreased in the presence of 5′-ethynylthymidine, a potent inhibitor of herpesvirus thymidine kinase, resulting in the abrogation of anti-KSHV activity of N-MCT. N-MCT-TP more effectively blocked in vitro DNA synthesis by KSHV DNA polymerase at IC₅₀ of 6.24±0.08 (mean±SD, μM) as compared to CDV-diphosphate (14.70±2.47) or GCV-TP (24.59±5.60). Taken together, N-MCT is a highly potent and target-specific anti-KSHV agent, which inhibits lytic KSHV DNA synthesis through its triphosphate metabolite produced in KSHV-infected cells expressing a virally encoded thymidine kinase. Other cyclopropanated carbocyclic 2′-deoxynucleosides can also be employed as anti-KS and anti-KSHV agents.

### Cyclopropanated Carbocyclic 2′-Deoxynucleosides

Carbocyclic 2′-deoxynucleoside analogs locked in the Northern conformation are effective agents in the prevention and treatment of KS-associated herpesvirus (KSHV, also called human herpesvirus 8 or HHV8) and KS. These compounds are described in U.S. Pat. No. 5,629,454 and in U.S. Pat. No. 5,869,666. Conformationally rigid (locked) nucleoside analogs are constructed on a bicyclo[3.1.0]hexane template whose value of P (pseudorotational angle) fits within the range of absolute Northern or Southern conformations. This bicyclo[3.1.0]hexane template exists exclusively as a pseudoboat, and carbocyclic nucleosides built thereon can adopt either a Northern or Southern conformation, depending on the relative disposition of substituents on the ring. Thus, a Northern C2′-exo (2E) envelope conformation is obtained when the cyclopropane ring was fused between carbon C4′ and the carbon supplanting the ribofuranoside oxygen. Conversely, fusion of the cyclopropane ring between carbon C1′ and the carbon supplanting the ribofuranoside oxygen provides the opposite Southern conformation. The cyclopropanated carbocyclic 2′-deoxynucleosides of preferred embodiments have the formula:

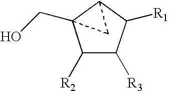

wherein R₁ is adenine, an adenine derivative, a substituted adenine, guanine, a guanine derivative, a substituted guanine, cytosine, a cytosine derivative, a substituted cytosine, thymine, a thymine derivative, a substituted thymine, uracil, a uracil derivative, or a substituted uracil; R₂ and R₃ are independently selected from hydrogen, allyl, alkylaryl, aryl, arylalkyl, alkoxy, alkyloxyalkyl, alkyloxyaryl, aryloxyalkyl, alkylaryloxy, aryloxy, and arylalkyloxy. If R₂ or R₃ is a moiety other than hydrogen, then it can be substituted, for example, by one or more halogen atoms. In a particularly preferred embodiment, the compounds exhibit the following stereochemistry:

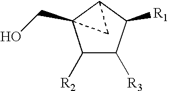

However, other stereochemistries can also be preferred.

The term “alkyl,” as used herein is a broad term and is used in its ordinary sense, including, without limitation, to refer to a straight chain or branched, acyclic or cyclic, unsaturated or saturated aliphatic hydrocarbon containing 1, 2, 3, 4, 5, 6, 7, 8, 9, or 10 or more carbon atoms, while the term “lower alkyl” has the same meaning as allyl but contains 1, 2, 3, 4, 5, or 6 carbon atoms. Representative saturated straight chain alkyls include methyl, ethyl, n-propyl, n-butyl, ii-pentyl, n-hexyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl,” respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1 butynyl, and the like. The term “cycloalkyl,” as used herein is a broad term and is used in its ordinary sense, including, without limitation, to refer to alkyls that include mono-, di-, or poly-homocyclic rings. Cycloalkyls are also referred to as “cyclic alkyls” or “homocyclic rings.” Representative saturated cyclic alkyls include cyclopropyl, cyclobutyl, cyclopentyl, cyclohexyl, —CH₂cyclopropyl, —CH₂cyclobutyl, —CH₂cyclopentyl, —CH₂cyclohexyl, and the like; while unsaturated cyclic alkyls include cyclopentenyl and cyclohexenyl, and the like. Cyclic alkyls include decalin, adamantane, and the like.

The term “aryl,” as used herein is a broad term and is used in its ordinary sense, including, without limitation, to refer to an aromatic carbocyclic moiety such as phenyl or naphthyl. The term “arylalkyl,” as used herein is a broad term and is used in its ordinary sense, including, without limitation, to refer to an alkyl having at least one alkyl hydrogen atom replaced with an aryl moiety, such as benzyl, —CH₂(1-naphthyl), —CH₂(2-naphthyl), —(CH₂)₂phenyl, —(CH₂)₃phenyl, —CH(phenyl)₂, and the like.

The term “substituted,” as used herein is a broad term and is used in its ordinary sense, including, without limitation, to refer to any of the above groups wherein at least one hydrogen atom is replaced with a substituent. In the case of a keto substituent (i.e., —C(═O)—) two hydrogen atoms are replaced. When substituted, “substituents,” within the context of preferred embodiments, include halogen, hydroxy, cyano, nitro, amino, alkylamino, dialkylamino, alkyl, alkoxy, alkylthio, haloalkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl, substituted heterocyclealkyl, —NRₐR_{b}, —NRₐC(═O)R_{b}, —NRₐC(═O)NR_{b}R_{c}, —NRₐC(═O)OR_{b}, —NRₐSO₂R_{b}, —ORₐ, —C(═O)Rₐ, —C(═O)ORₐ, —C(═O)NRₐR_{b}, —SH, —SRₐ, —SORₐ, —S(═O)₂Rₐ, —OS(═O)₂Rₐ, —OC(═O)NRₐR_{b}, —S(═O)₂ORₐ, wherein Rₐ, R_{b}, and R_{c} are the same or different and are independently selected from hydrogen, allyl, haloalkyl, substituted alkyl, aryl, substituted aryl, arylalkyl, substituted arylalkyl, heteroaryl, substituted heteroaryl, heteroarylalkyl, substituted heteroarylalkyl, heterocycle, substituted heterocycle, heterocyclealkyl or substituted heterocyclealkyl.

The term “halogen,” as used herein is a broad term and is used in its ordinary sense, including, without limitation, to refer to fluoro, chloro, bromo, and iodo. The term “haloalkyl,” as used herein is a broad term and is used in its ordinary sense, including, without limitation, to refer to an alkyl having at least one hydrogen atom replaced with halogen, such as trifluoromethyl and the like. The term “alkoxy,” as used herein is a broad term and is used in its ordinary sense, including, without limitation, to refer to an allyl moiety attached through an oxygen bridge (i.e., —O-alkyl) such as methoxy, ethoxy, and the like. The term “hydroxyalkyl” as used herein is a broad term and is used in its ordinary sense, including, without limitation, to refer to an alkyl substituted with at least one hydroxyl group. The term “mono- or di-(cycloalkyl)methyl,” as used herein is a broad term and is used in its ordinary sense, including, without limitation, to refer to a methyl group substituted with one or two cycloalkyl groups, such as cyclopropylmethyl, dicyclopropylmethyl, and the like.

The term “alkyloxyalkyl” as used herein is a broad term and is used in its ordinary sense, including, without limitation, to refer to an allyl substituted with an -alkyl group. The cyclic systems referred to herein include fused ring, bridged ring, and spiro ring moieties, in addition to isolated monocyclic moieties.

Preferred cyclopropanated carbocyclic 2′-deoxynucleosides include (N)-2′-deoxy-methanocarba-A (adenosine analog), (N)-methanocarba-T (thymidine analog), (N)-2′-deoxy-methanocarba-G (guanosine analog), (N)-2′-deoxy-methanocarba-C (cytosine analog) and (N)-2′-deoxy-methanocarba-U (uridine analog). These particular cyclopropanated carbocyclic 2′-deoxynucleosides are depicted by the following structure:

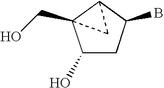

wherein B is adenine, thymine, cytosine, guanine or uracil.

The thymine analogs, especially North-methanocarbathymidine (N-MCT), are particularly preferred because of their exceptionally potent activity against KSHV and KS.

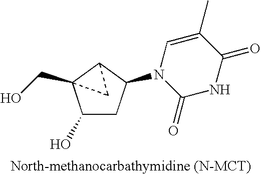

The synthesis of the (N)-methanocarbathymidine (or its adenosine, guanine, cytidine, or uridine analogs) is described below and in Schemes 1-4. The anti-KSHV effect of various substituted derivatives of the (N)-methanocarba 2′-deoxynucleoside analogs described below can easily be determined by one of ordinary skill in the art using the assay methods described herein without undue experimentation.

Schemes 1-2 can be utilized for the synthesis of intermediate 12, which is chiral, so there is no need for optical resolution at the end of the synthesis, and which can be employed as a starting material for the synthesis of related carbocyclic 2′-deoxynucleoside analogs. Cyclopentenol 6 can be obtained from the sodium borohydride reduction of cyclopentenone 5 (Marquez et al., J. Org. Chem., 53:5709, 1988). Regioselective cleavage of the contiguous O-isopropylidenetriol system in 6 with trimethylaluminum (Takano et al., Tetrahedron Lett., 29:1823, 1988) produces the corresponding carbocyclic 3-tert-butoxy-1,5-glycol 7, which in the presence of tert-butyldimethylsilyl chloride reacts exclusively at the less hindered allylic alcohol position to give the protected intermediate 8. Barton's radical deoxygenation of 8 at C-5 occurs via the xanthate 9 in the presence of AIBN to give compound 10. Deprotection of the silyl ether in 10 by fluoride ion unmasks the hydroxyl group (compound 11) which directs the ensuing cyclopropanation to give compound 12.

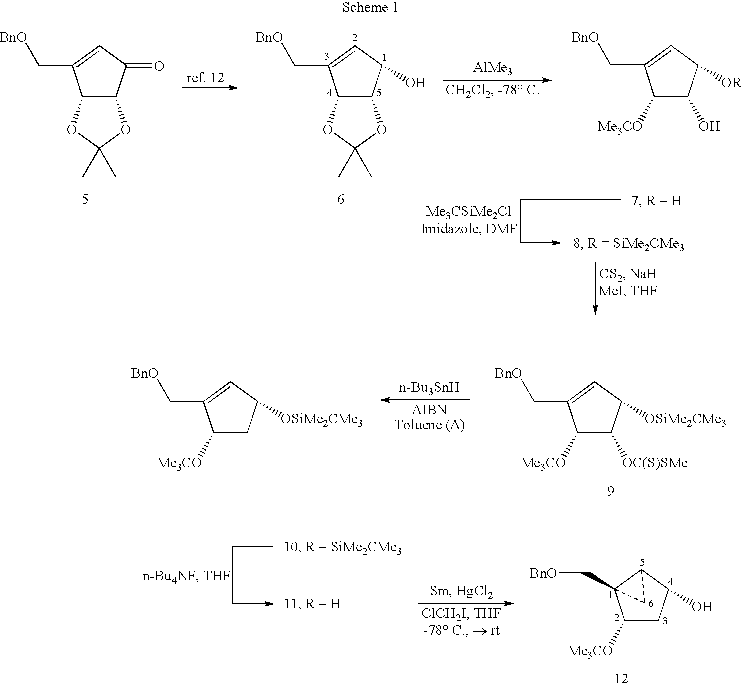

This compound is directly coupled to 6-chloropurine under Mitsunobu conditions (Mitsunobu, 1981 Synthesis 1:1-28) to give the protected carbocyclic nucleoside intermediate 13. Following aminolysis of 13 with ammonia, and the simultaneous removal of both benzyl and tert-butyl groups, the (N)-2′-deoxy-methanocarba adenosine derivative 4 is obtained.

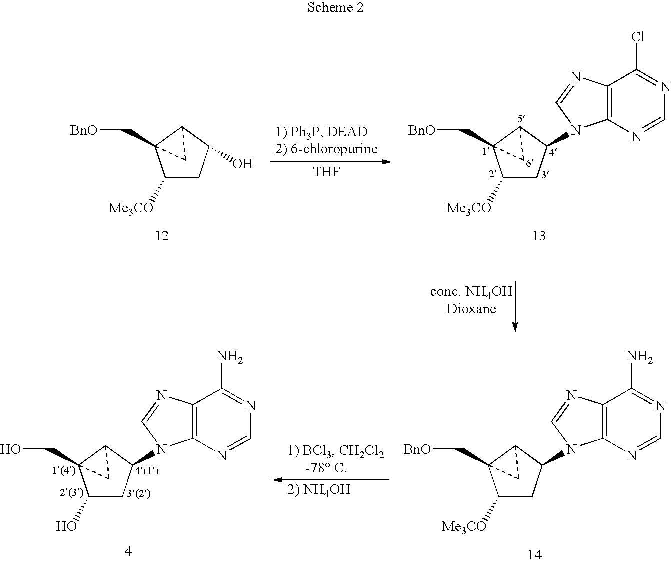

For the pyrimidine derivatives (Scheme 3), protected N³-benzoylthymine and N³-benzoyluracil (Cruickshank et al. 1994 Tetrahedron Lett 25:681) are coupled according to Scheme 3. In the case of 16, the O-alkylated product predominates, whereas for the uracil analog 17, the situation is reversed. Base-catalyzed deprotection of the N-benzoyl group from intermediates 16 and 17 yields the penultimate intermediates 18 and 19, respectively, and simultaneous removal of both O-benzyl and O-tert-butyl groups with BCl₃ provide the desired targets (N)-methanocarba-T 20 and (N)-methanocarba-U 21. (N)-methanocarba-C 22 is prepared from (N)-methanocarba-U 21 via formation of the triazole intermediate (Divakar et al. 1982 J Chem Soc Perkin Trans I:1171-1176, 1982).

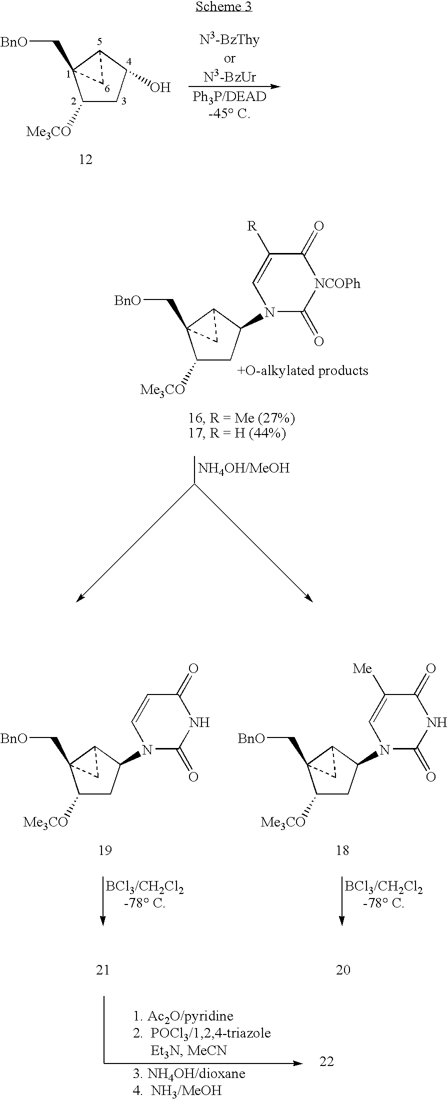

For the synthesis of (N)-methanocarba-G 24 (Scheme 4), coupling under Mitsunobu conditions proceeds with a yield comparable to that of the pyrimidines. Only the desired N-9 isomer (34%) is obtained with virtually no detection of the N-7 isomer. The conversion of the 2-amino-6-chloro intermediate into the 6-O-benzyl derivative 23 facilitates the one-step removal of all protective groups in the generation of the guanine base (Rodriguez et al. 1993 Tetrahedron Lett 34:6233-6236; Rodriguez et al. 1994 J Med Chem 37:3389-3399).

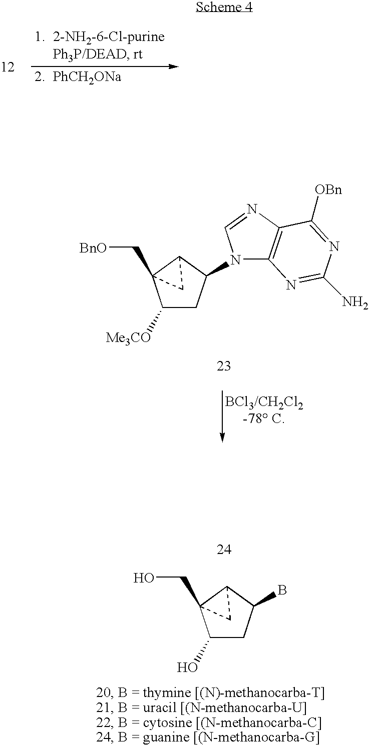

The cyclopropanated carbocyclic 2′-deoxynucleosides of preferred embodiments can also be incorporated into short oligodeoxynucleotides (ODNs). Standard double helices exist in the classic B-DNA form, in which all sugars have a Southern conformation, or in the A-DNA form, wherein the sugars have a N-conformation. During formation of DNA/RNA heteroduplexes, the A-form, typical of RNA, is dominant. The expected thermodynamic stability resulting from the preorganization of the pseudosugar rings into the Northern conformation, typical of A-DNA, is evident by the increase in melting temperature (Tₘ) of the corresponding DNA/RNA heteroduplex containing the (N)-methanocarba T.

### Pharmaceutical Compositions Comprising Cyclopropanated Carbocyclic 2′-Deoxynucleosides

The cyclopropanated carbocyclic 2′-deoxynucleosides (or derivatives, nucleoside prodrugs, or pharmaceutically acceptable esters or salts thereof) of the preferred embodiments, can be incorporated into a pharmaceutically acceptable carrier for administration to an individual having a KSHV infection, having KS, or can be administered prophylactically to prevent KSHV infection or KS. The cyclopropanated carbocyclic 2′-deoxynucleoside can be employed as the sole agent in the prevention or treatment of KSHV or KS, or two or more cyclopropanated carbocyclic 2′-deoxynucleosides can be employed, optionally in combination with other therapeutic agents, e.g., drugs employed in the treatment of KSHV or KS, other viral infections, such as AIDS or HIV, or cancer.

The terms “pharmaceutically acceptable salts” and “a pharmaceutically acceptable salt thereof” as used herein are broad terms and are used in their ordinary sense, including, without limitation, to refer to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the cyclopropanated carbocyclic 2′-deoxynucleoside or other therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower allyl esters, and protected derivatives can also be suitable for use in compositions and methods of preferred embodiments.

Contemplated routes of administration include topical, oral, intravenous, subcutaneous, parenteral, intradermal, intramuscular, intraperitoneal, intraocular, and intravenous, including injectable administration, sustained release from implants, administration by eyedrops, and the like. Nonlimiting examples of particularly preferred nucleoside analog compositions for topical administration include creams, lotions, gels, salves, sprays, dispersions, suspensions, pastes, and ointments.

The cyclopropanated carbocyclic 2′-deoxynucleosides of preferred embodiments can be formulated into liquid preparations for, e.g., oral, nasal, anal, rectal, buccal, vaginal, peroral, intragastric, mucosal, perlingual, alveolar, gingival, olfactory, or respiratory mucosa administration. Suitable forms for such administration include suspensions, syrups, and elixirs. If nasal or respiratory (mucosal) administration is desired (e.g., aerosol inhalation or insufflation), compositions may be in a form and dispensed by a squeeze spray dispenser, pump dispenser or aerosol dispenser. Aerosols are usually under pressure by means of a hydrocarbon. Pump dispensers can preferably dispense a metered dose or a dose having a particular particle size.

The pharmaceutical compositions containing cyclopropanated carbocyclic 2′-deoxynucleosides are preferably isotonic with the blood or other body fluid of the recipient. The isotonicity of the compositions can be attained using sodium tartrate, propylene glycol or other inorganic or organic solutes. Sodium chloride is particularly preferred. Buffering agents can be employed, such as acetic acid and salts, citric acid and salts, boric acid and salts, and phosphoric acid and salts. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like.

Viscosity of the pharmaceutical compositions can be maintained at the selected level using a pharmaceutically acceptable thickening agent. Methylcellulose is preferred because it is readily and economically available and is easy to work with. Other suitable thickening agents include, for example, xanthan gum, carboxymethyl cellulose, hydroxypropyl cellulose, carbomer, and the like. The preferred concentration of the thickener will depend upon the thickening agent selected. An amount is preferably used that will achieve the selected viscosity. Viscous compositions are normally prepared from solutions by the addition of such thickening agents.

A pharmaceutically acceptable preservative can be employed to increase the shelf life of the pharmaceutical compositions. Benzyl alcohol can be suitable, although a variety of preservatives including, for example, parabens, thimerosal, chlorobutanol, or benzalkonium chloride can also be employed. A suitable concentration of the preservative is typically from about 0.02% to about 2% based on the total weight of the composition, although larger or smaller amounts can be desirable depending upon the agent selected.

The cyclopropanated carbocyclic 2′-deoxynucleosides of preferred embodiments can be in admixture with a suitable carrier, diluent, or excipient such as sterile water, physiological saline, glucose, or the like, and can contain auxiliary substances such as wetting or emulsifying agents, pH buffering agents, gelling or viscosity enhancing additives, preservatives, flavoring agents, colors, and the like, depending upon the route of administration and the preparation desired. Standard texts, such as “Remington: The Science and Practice of Pharmacy”, Lippincott Williams & Wilkins; 20th edition (Jun. 1, 2003) and “Remington's Pharmaceutical Sciences,” Mack Pub. Co.; 18^{th} and 19^{th} editions (December 1985, and June 1990, respectively). Such preparations can include complexing agents, metal ions, polymeric compounds such as polyacetic acid, polyglycolic acid, hydrogels, dextran, and the like, liposomes, microemulsions, micelles, unilamellar or multilamellar vesicles, erythrocyte ghosts or spheroblasts. Suitable lipids for liposomal formulation include, without limitation, monoglycerides, diglycerides, sulfatides, lysolecithin, phospholipids, saponin, bile acids, and the like. The presence of such additional components can influence the physical state, solubility, stability, rate of in vivo release, and rate of in vivo clearance, and are thus chosen according to the intended application, such that the characteristics of the carrier are tailored to the selected route of administration.

For oral administration, the cyclopropanated carbocyclic 2′-deoxynucleosides can be provided as a tablet, aqueous or oil suspension, dispersible powder or granule, emulsion, hard or soft capsule, syrup or elixir. Compositions intended for oral use can be prepared according to any method known in the art for the manufacture of pharmaceutical compositions and can include one or more of the following agents: sweeteners, flavoring agents, coloring agents and preservatives. Aqueous suspensions can contain the active ingredient in admixture with excipients suitable for the manufacture of aqueous suspensions.

Formulations for oral use can also be provided as hard gelatin capsules, wherein the cyclopropanated carbocyclic 2′-deoxynucleoside is mixed with an inert solid diluent, such as calcium carbonate, calcium phosphate, or kaolin, or as soft gelatin capsules. In soft capsules, the active compounds can be dissolved or suspended in suitable liquids, such as water or an oil medium, such as peanut oil, olive oil, fatty oils, liquid paraffin, or liquid polyethylene glycols. Stabilizers and microspheres formulated for oral administration can also be used. Capsules can include push-fit capsules made of gelatin, as well as soft, sealed capsules made of gelatin and a plasticizer, such as glycerol or sorbitol. The push-fit capsules can contain the cyclopropanated carbocyclic 2′-deoxynucleoside in admixture with fillers such as lactose, binders such as starches, and/or lubricants such as talc or magnesium stearate and, optionally, stabilizers.

Tablets can be uncoated or coated by known methods to delay disintegration and absorption in the gastrointestinal tract and thereby provide a sustained action over a longer period of time. For example, a time delay material such as glyceryl monostearate can be used. When administered in solid form, such as tablet form, the solid form typically comprises from about 0.001 wt. % or less to about 50 wt. % or more of active ingredient(s) including the cyclopropanated carbocyclic 2′-deoxynucleoside, preferably from about 0.005, 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, or 1 wt. % to about 2, 3, 4, 5, 6, 7, 8, 9, 10, 15, 20, 25, 30, 35, 40, or 45 wt. %.

Tablets can contain the cyclopropanated carbocyclic 2′-deoxynucleoside in admixture with non-toxic pharmaceutically acceptable excipients including inert materials. For example, a tablet can be prepared by compression or molding, optionally, with one or more additional ingredients. Compressed tablets can be prepared by compressing in a suitable machine the active ingredients in a free-flowing form such as powder or granules, optionally mixed with a binder, lubricant, inert diluent, surface active or dispersing agent. Molded tablets can be made by molding, in a suitable machine, a mixture of the powdered compound moistened with an inert liquid diluent.

Preferably, each tablet or capsule contains from about 10 mg or less to about 1,000 mg or more of the cyclopropanated carbocyclic 2′-deoxynucleoside, more preferably from about 20, 30, 40, 50, 60, 70, 80, 90, or 100 mg to about 150, 200, 250, 300, 350, 400, 450, 500, 550, 600, 650, 700, 750, 800, or 900 mg. Most preferably, tablets or capsules are provided in a range of dosages to permit divided dosages to be administered. A dosage appropriate to the patient and the number of doses to be administered daily can thus be conveniently selected. While it is generally preferred to incorporate the cyclopropanated carbocyclic 2′-deoxynucleoside and any other therapeutic agent employed in combination therewith in a single tablet or other dosage form, e.g., in a combination therapy, in certain embodiments it can be desirable to provide the cyclopropanated carbocyclic 2′-deoxynucleoside and other therapeutic agents in separate dosage forms.

Suitable inert materials include diluents, such as carbohydrates, mannitol, lactose, anhydrous lactose, cellulose, sucrose, modified dextrans, starch, and the like, or inorganic salts such as calcium triphosphate, calcium phosphate, sodium phosphate, calcium carbonate, sodium carbonate, magnesium carbonate, and sodium chloride. Disintegrants or granulating agents can be included in the formulation, for example, starches such as corn starch, alginic acid, sodium starch glycolate, Amberlite, sodium carboxymethylcellulose, ultramylopectin, sodium alginate, gelatin, orange peel, acid carboxymethyl cellulose, natural sponge and bentonite, insoluble cationic exchange resins, powdered gums such as agar, karaya or tragacanth, or alginic acid or salts thereof.

Binders can be used to form a hard tablet. Binders include materials from natural products such as acacia, tragacanth, starch and gelatin, methyl cellulose, ethyl cellulose, carboxymethyl cellulose, polyvinyl pyrrolidone, hydroxypropylmethyl cellulose, and the like.

Lubricants, such as stearic acid or magnesium or calcium salts thereof, polytetrafluoroethylene, liquid paraffin, vegetable oils and waxes, sodium lauryl sulfate, magnesium lauryl sulfate, polyethylene glycol, starch, talc, pyrogenic silica, hydrated silicoaluminate, and the like, can be included in tablet formulations.

Surfactants can also be employed, for example, anionic detergents such as sodium lauryl sulfate, dioctyl sodium sulfosuccinate and dioctyl sodium sulfonate, cationic such as benzalkonium chloride or benzethonium chloride, or nonionic detergents such as polyoxyethylene hydrogenated castor oil, glycerol monostearate, polysorbates, sucrose fatty acid ester, methyl cellulose, or carboxymethyl cellulose.

Controlled release formulations can be employed wherein the cyclopropanated carbocyclic 2′-deoxynucleoside is incorporated into an inert matrix that permits release by either diffusion or leaching mechanisms. Slowly degenerating matrices can also be incorporated into the formulation. Other delivery systems can include timed release, delayed release, or sustained release delivery systems.

Coatings can be used, for example, nonenteric materials such as methyl cellulose, ethyl cellulose, hydroxyethyl cellulose, methylhydroxy-ethyl cellulose, hydroxypropyl cellulose, hydroxypropyl-methyl cellulose, sodium carboxy-methyl cellulose, providone and the polyethylene glycols, or enteric materials such as phthalic acid esters. Dyestuffs or pigments can be added for identification or to characterize different combinations of active compound doses

When administered orally in liquid form, a liquid carrier such as water, petroleum, oils of animal or plant origin such as peanut oil, mineral oil, soybean oil, or sesame oil, or synthetic oils can be added to the cyclopropanated carbocyclic 2′-deoxynucleoside. Physiological saline solution, dextrose, or other saccharide solution, or glycols such as ethylene glycol, propylene glycol, or polyethylene glycol are also suitable liquid carriers. The pharmaceutical compositions can also be in the form of oil-in-water emulsions. The oily phase can be a vegetable oil, such as olive or arachis oil, a mineral oil such as liquid paraffin, or a mixture thereof. Suitable emulsifying agents include naturally-occurring gums such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsions can also contain sweetening and flavoring agents.

Pulmonary delivery of the cyclopropanated carbocyclic 2′-deoxynucleosides of preferred embodiments can also be employed. The cyclopropanated carbocyclic 2′-deoxynucleoside is delivered to the lungs while inhaling and traverses across the lung epithelial lining to the blood stream. A wide range of mechanical devices designed for pulmonary delivery of therapeutic products can be employed, including but not limited to nebulizers, metered dose inhalers, and powder inhalers, all of which are familiar to those skilled in the art. These devices employ formulations suitable for the dispensing of the cyclopropanated carbocyclic 2′-deoxynucleoside. Typically, each formulation is specific to the type of device employed and can involve the use of an appropriate propellant material, in addition to diluents, adjuvants, and/or carriers useful in therapy.

The cyclopropanated carbocyclic 2′-deoxynucleoside and other optional active ingredients are advantageously prepared for pulmonary delivery in particulate form with an average particle size of from 0.1 μm or less to 10 μm or more, more preferably from about 0.2, 0.3, 0.4, 0.5, 0.6, 0.7, 0.8, or 0.9 μm to about 1.0, 1.5, 2.0, 2.5, 3.0, 3.5, 4.0, 4.5, 5.0, 5.5, 6.0, 6.5, 7.0, 7.5, 8.0, 8.5, 9.0, or 9.5 μm. Pharmaceutically acceptable carriers for pulmonary delivery of the cyclopropanated carbocyclic 2′-deoxynucleosides include carbohydrates such as trehalose, mannitol, xylitol, sucrose, lactose, and sorbitol. Other ingredients for use in formulations can include DPPC, DOPE, DSPC, and DOPC. Natural or synthetic surfactants can be used, including polyethylene glycol and dextrans, such as cyclodextran. Bile salts and other related enhancers, as well as cellulose and cellulose derivatives, and amino acids can also be used. Liposomes, microcapsules, microspheres, inclusion complexes, and other types of carriers can also be employed.

Pharmaceutical formulations suitable for use with a nebulizer, either jet or ultrasonic, typically comprise the cyclopropanated carbocyclic 2′-deoxynucleoside dissolved or suspended in water at a concentration of about 0.01 or less to 100 mg or more of cyclopropanated carbocyclic 2′-deoxynucleoside per mL of solution, preferably from about 0.1, 1, 2, 3, 4, 5, 6, 7, 8, 9, or 10 mg to about 15, 20, 25, 30, 35, 40, 45, 50, 55, 60, 65, 70, 75, 80, 85, or 90 mg per mL of solution. The formulation can also include a buffer and a simple sugar (e.g., for protein stabilization and regulation of osmotic pressure). The nebulizer formulation can also contain a surfactant, to reduce or prevent surface induced aggregation of the cyclopropanated carbocyclic 2′-deoxynucleoside caused by atomization of the solution in forming the aerosol.

Formulations for use with a metered-dose inhaler device generally comprise a finely divided powder containing the active ingredients suspended in a propellant with the aid of a surfactant. The propellant can include conventional propellants, such as chlorofluorocarbons, hydrochlorofluorocarbons, hydrofluorocarbons, and hydrocarbons. Preferred propellants include trichlorofluoromethane, dichlorodifluoromethane, dichlorotetrafluoroethanol, 1,1,1,2-tetrafluoroethane, and combinations thereof. Suitable surfactants include sorbitan trioleate, soya lecithin, and oleic acid.

Formulations for dispensing from a powder inhaler device typically comprise a finely divided dry powder containing the cyclopropanated carbocyclic 2′-deoxynucleoside, optionally including a bulking agent, such as lactose, sorbitol, sucrose, mannitol, trehalose, or xylitol in all amount that facilitates dispersal of the powder from the device, typically from about 1 wt. % or less to 99 wt. % or more of the formulation, preferably from about 5, 10, 15, 20, 25, 30, 35, 40, 45, or 50 wt. % to about 55, 60, 65, 70, 75, 80, 85, or 90 wt. % of the formulation.

When the cyclopropanated carbocyclic 2′-deoxynucleoside is administered by intravenous, cutaneous, subcutaneous, parenteral, or other injection, it is preferably in the form of a pyrogen-free, parenterally acceptable aqueous solution or oleaginous suspension. Suspensions can be formulated according to methods well known in the art using suitable dispersing or wetting agents and suspending agents. The preparation of acceptable aqueous solutions with suitable pH, isotonicity, stability, and the like, is within the skill in the art. A preferred pharmaceutical composition for injection preferably contains an isotonic vehicle such as 1,3-butanediol, water, isotonic sodium chloride solution, Ringer's solution, dextrose solution, dextrose and sodium chloride solution, lactated Ringer's solution, or other vehicles as are known in the art. In addition, sterile fixed oils can be employed conventionally as a solvent or suspending medium. For this purpose, any bland fixed oil can be employed including synthetic mono or diglycerides. In addition, fatty acids such as oleic acid can likewise be used in the formation of injectable preparations. The pharmaceutical compositions can also contain stabilizers, preservatives, buffers, antioxidants, or other additives known to those of skill in the art.

The duration of the injection can be adjusted depending upon various factors, and can comprise a single injection administered over the course of a few seconds or less, to 1, 2, 3, 4, 5, 6, 7, 8, 9, 10, 11, 12, 13, 14, 15, 16, 17, 18, 19, 20, 21, 22, 23, or 24 hours or more of continuous intravenous administration.

The cyclopropanated carbocyclic 2′-deoxynucleosides can be administered systematically or locally, via a liquid or gel, or as an implant or device.

The anti-KSHV and anti-KS compositions of the preferred embodiments can additionally employ adjunct components conventionally found in pharmaceutical compositions in their art-established fashion and at their art-established levels. Thus, for example, the compositions can contain additional compatible pharmaceutically active materials for combination therapy (such as supplementary antimicrobials, antipruritics, astringents, local anesthetics, anticancer, or anti-inflammatory agents), or can contain materials useful in physically formulating various dosage forms of the preferred embodiments, such as excipients, dyes, perfumes, thickening agents, stabilizers, skin penetration enhancers, preservatives or antioxidants.

The cyclopropanated carbocyclic 2′-deoxynucleosides of preferred embodiments are particularly well suited for use in preparations including other therapeutic agents, for example, anti-microbials agents such as anti-bacterials, anti-mycobacterials, anti-virals (e.g., as approved for the treatment of HIV infection), anti-fungal, and anti-parasites. Examples of anti-bacterials include beta-lactam antibiotics, penicillins (such as natural penicillins, aminopenicillins, penicillinase-resistant penicillins, carboxy penicillins, ureido penicillins), cephalosporins (first generation, second generation, and third generation cephalosporins), and other beta-lactams (such as imipenem, monobactams), beta-lactamase inhibitors, vancomycin, aminoglycosides and spectinomycin, tetracyclines, chloramphenicol, erythromycin, lincomycin, clindamycin, rifampin, metronidazole, polymyxins, sulfonamides and trimethoprim, and quinolines, Acedapsone; Acetosulfone Sodium; Alamecin; Alexidine; Amdinocillin; Amdinocillin Pivoxil; Amicycline; Amifloxacin; Amifloxacin Mesylate; Amikacin; Amikacin Sulfate; Aminosalicylic acid; Aminosalicylate sodium; Amoxicillin; Amphomycin; Ampicillin; Ampicillin Sodium; Apalcillin Sodium; Apramycin; Aspartocin; Astromicin Sulfate; Avilamycin; Avoparcin; Azithromycin; Azlocillin; Azlocillin Sodium; Bacampicillin Hydrochloride; Bacitracin; Bacitracin Methylene Disalicylate; Bacitracin Zinc; Bambermycins; Benzoylpas Calcium; Berythromycin; Betamicin Sulfate; Biapenem; Biniramycin; Biphenamine Hydrochloride; Bispyrithione Magsulfex; Butikacin; Butirosin Sulfate; Capreomycin Sulfate; Carbadox; Carbenicillin Disodium; Carbenicillin Indanyl Sodium; Carbenicillin Phenyl Sodium; Carbenicillin Potassium; Carumonam Sodium; Cefaclor; Cefadroxil; Cefamandole; Cefamandole Nafate; Cefamandole Sodium; Cefaparole; Cefatrizine; Cefazaflur Sodium; Cefazolin; Cefazolin Sodium; Cefbuperazone; Cefdinir; Cefepime; Cefepime Hydrochloride; Cefetecol; Cefixime; Cefinenoxime Hydrochloride; Cefinetazole; Cefinetazole Sodium; Cefonicid Monosodium; Cefonicid Sodium; Cefoperazone Sodium; Ceforanide; Cefotaxime Sodium; Cefotetan; Cefotetan Disodium; Cefotiam Hydrochloride; Cefoxitin; Cefoxitin Sodium; Cefpimizole; Cefpimizole Sodium; Cefpiramide; Cefpiramide Sodium; Cefpirome Sulfate; Cefpodoxime Proxetil; Cefprozil; Cefroxadine; Cefsulodin Sodium; Ceftazidime; Ceftibuten; Ceftizoxime Sodium; Ceftriaxone Sodium; Cefuroxime; Cefuroxime Axetil; Cefuroxime Pivoxetil; Cefuroxime Sodium; Cephacetrile Sodium; Cephalexin; Cephalexin Hydrochloride; Cephaloglycin; Cephaloridine; Cephalothin Sodium; Cephapirin Sodium; Cephradine; Cetocycline Hydrochloride; Cetophenicol; Chloramphenicol; Chloramphenicol Palmitate; Chloramphenicol Pantothenate Complex; Chloramphenicol Sodium Succinate; Chlorhexidine Phosphanilate; Chloroxylenol; Chlortetracycline Bisulfate; Chlortetracycline Hydrochloride; Cinoxacin; Ciprofloxacin; Ciprofloxacin Hydrochloride; Cirolemycin; Claritlhromycin; Clinafloxacin Hydrochloride; Clindamycin; Clindamycin Hydrochloride; Clindamycin Palmitate Hydrochloride; Clindamycin Phosphate; Clofazimine; Cloxacillin Benzathine; Cloxacillin Sodium; Cloxyquin; Colistimethate Sodium; Colistin Sulfate; Coumermycin; Coumermycin Sodium; Cyclacillin; Cycloserine; Dalfopristin; Dapsone; Daptomycin; Demeclocycline; Demeclocycline Hydrochloride; Demecycline; Denofungin; Diaveridine; Dicloxacillin; Dicloxacillin Sodium; Dihydrostreptomycin Sulfate; Dipyrithione; Dirithromycin; Doxycycline; Doxycycline Calcium; Doxycycline Fosfatex; Doxycycline Hyclate; Droxacin Sodium; Enoxacin; Epicillin; Epitetracycline Hydrochloride; Erythromycin; Erythromycin Acistrate; Erythromycin Estolate; Erythromycin Ethylsuccinate; Erythromycin Gluceptate; Erythromycin Lactobionate; Erythromycin Propionate; Erythromycin Stearate; Ethambutol Hydrochloride; Ethionamide; Fleroxacin; Floxacillin; Fludalanine; Flumequine; Fosfomycin; Fosfomycin Tromethamine; Fumoxicillin; Furazolium Chloride; Furazolium Tartrate; Fusidate Sodium; Fusidic Acid; Gentamicin Sulfate; Gloximonam; Gramicidin; Haloprogin; Hetacillin; Hetacillin Potassium; Hexedine; Ibafloxacin; Imipenem; Isoconazole; Isepamicin; Isoniazid; Josamycin; Kanamycin Sulfate; Kitasamycin; Levofuraltadone; Levopropylcillin Potassium; Lexithromycin; Lincomycin; Lincomycin Hydrochloride; Lomefloxacin; Lomefloxacin Hydrochloride; Lomefloxacin Mesylate; Loracarbef; Mafenide; Meclocycline; Meclocycline Sulfosalicylate; Megalomicin Potassium Phosphate; Mequidox; Meropenem; Methacycline; Methacycline Hydrochloride; Methenamine; Methenamine Hippurate; Methenamine Mandelate; Methicillin Sodium; Metioprim; Metronidazole Hydrochloride; Metronidazole Phosphate; Mezlocillin; Meziocillin Sodium; Minocycline; Minocycline Hydrochloride; Mirincamycin Hydrochloride; Monensin; Monensin Sodium; Nafcillin Sodium; Nalidixate Sodium; Nalidixic Acid; Natamycin; Nebramycin; Neomycin Palmitate; Neomycin Sulfate; Neomycin Undecylenate; Netilmicin Sulfate; Neutramycin; Nifuradene; Nifuraldezone; Nifuratel; Nifuratrone; Nifurdazil; Nifurimide; Nifurpirinol; Nifurquinazol; Nifurthiazole; Nitrocycline; Nitrofurantoin; Nitromide; Norfloxacin; Novobiocin Sodium; Ofloxacin; Ormetoprim; Oxacillin Sodium; Oximonam; Oximonam Sodium; Oxolinic Acid; Oxytetracycline; Oxytetracycline Calcium; Oxytetracycline Hydrochloride; Paldimycin; Parachlorophenol; Paulomycin; Pefloxacin; Pefloxacin Mesylate; Penamecillin; Penicillin G Benzathine; Penicillin G Potassium; Penicillin G Procaine; Penicillin G Sodium; Penicillin V; Penicillin V Benzathine; Penicillin V Hydrabamine; Penicillin V Potassium; Pentizidone Sodium; Phenyl Aminosalicylate; Piperacillin Sodium; Pirbenicillin Sodium; Piridicillin Sodium; Pirlimycin Hydrochloride; Pivampicillin Hydrochloride; Pivampicillin Pamoate; Pivampicillin Probenate; Polymyxin B Sulfate; Porfiromycin; Propikacin; Pyrazinamide; Pyrithione Zinc; Quindecamine Acetate; Quinupristin; Racephenicol; Ramoplanin; Ranimycin; Relomycin; Repromicin; Rifabutin; Rifametane; Rifamexil; Rifamide; Rifampin; Rifapentine; Rifaximin; Rolitetracycline; Rolitetracycline Nitrate; Rosaramicin; Rosaramicin Butyrate; Rosaramicin Propionate; Rosaramicin Sodium Phosphate; Rosaramicin Stearate; Rosoxacin; Roxarsone; Roxithromycin; Sancycline; Sanfetrinem Sodium; Sarmoxicillin; Sarpicillin; Scopafungin; Sisomicin; Sisomicin Sulfate; Sparfloxacin; Spectinomycin Hydrochloride; Spiramycin; Stallimycin Hydrochloride; Steffimycin; Streptomycin Sulfate; Streptonicozid; Sulfabenz; Sulfabenzamide; Sulfacetamide; Sulfacetamide Sodium; Sulfacytine; Sulfadiazine; Sulfadiazine Sodium; Sulfadoxine; Sulfalene; Sulfamerazine; Sulfameter; Sulfamethazine; Sulfamethizole; Sulfamethoxazole; Sulfamonomethoxine; Sulfamoxole; Sulfanilate Zinc; Sulfanitran; Sulfasalazine; Sulfasomizole; Sulfathiazole; Sulfazamet; Sulfisoxazole; Sulfisoxazole Acetyl; Sulfisoxazole Diolamine; Sulfomyxin; Sulopenem; Sultamicillin; Suncillin Sodium; Talampicillin Hydrochloride; Teicoplanin; Temafloxacin Hydrochloride; Temocillin; Tetracycline; Tetracycline Hydrochloride; Tetracycline Phosphate Complex; Tetroxoprim; Thiamphenicol; Thiphencillin Potassium; Ticarcillin Cresyl Sodium; Ticarcillin Disodium; Ticarcillin Monosodium; Ticlatone; Tiodonium Chloride; Tobramycin; Tobramycin Sulfate; Tosufloxacin; Trimethoprim; Trimethoprim Sulfate; Trisulfapyrimidines; Troleandomycin; Trospectomycin Sulfate; Tyrothricin; Vancomycin; Vancomycin Hydrochloride; Virginiamycin; Zorbamycin. Anti-mycobacterials include Myambutol (Ethambutol Hydrochloride), Dapsone (4,4′-diaminodiphenylsulfone), Paser Granules (aminosalicylic acid granules), Priftin (rifapentine), Pyrazinamide, Isoniazid, Rifadin (Rifampin), Rifadin IV, Rifamate (Rifampin and Isoniazid), Rifater (Rifampin, Isoniazid, and Pyrazinamide), Streptomycin Sulfate and Trecator-SC (Ethionamide). Anti-virals include amantidine, rimantadine, ribivarin, acyclovir, delavirdine, efavirenz, enfuvirtide, ritonavir, indinavir, nelfinavir, saquinavir, lopinavir, atazanavir, fosamprenavir, tipranavir, abacavir, tenofovir disoproxil fumarate, emtricitabine, vidarabine, trifluorothymidine, ganciclovir, zidovudine, retinovir, interferons, Acemannan; Acyclovir; Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; Zinviroxime and integrase inhibitors. Anti-fungals include imidazoles and triazoles, polyene macrolide antibiotics, griseofulvin, amphotericin B, and flucytosine. Antiparasites include heavy metals, antimalarial quinolines, folate antagonists, nitroimidazoles, benzimidazoles, avermectins, praxiquantel, ornithine decarboxylase inhibitors, phenols (e.g., bithionol, niclosamide); synthetic alkaloid (e.g., dehydroemetine); piperazines (e.g., diethylcarbamazine); acetanilide (e.g., diloxanide furonate); halogenated quinolines (e.g., iodoquinol (diiodohydroxyquin)); nitrofurans (e.g., nifurtimox); diamidines (e.g., pentamidine); tetrahydropyrimidine (e.g., pyrantel pamoate); sulfated naphthylamine (e.g., suramin).

Preferred anti-infectives for use in combination with the cyclopropanated carbocyclic 2′-deoxynucleosides of preferred embodiments include Difloxacin Hydrochloride; Lauryl Isoquinolinium Bromide; Moxalactarn Disodium; Omidazole; Pentisomicin; Sarafloxacin Hydrochloride; Protease inhibitors of HIV and other retroviruses; Integrase inhibitors of HIV and other retroviruses; Cefaclor (Ceclor); Acyclovir (Zovirax); Norfloxacin (Noroxin); Cefoxitin (Mefoxin); Cefuroxime axetil (Ceftin); Ciprofloxacin (Cipro); Aminacrine Hydrochloride; Benzethonium Chloride: Bithionolate Sodium; Bromchlorenone; Carbamide Peroxide; Cetalkonium Chloride; Cetylpyridinium Chloride Chlorhexidine Hydrochloride; Clioquinol; Domiphen Bromide; Fenticlor; Fludazonium Chloride; Fuchsin, Basic; Furazolidone; Gentian Violet; Halquinols; Hexachlorophene: Hydrogen Peroxide; Ichthammol; Imidecyl Iodine; Iodine; Isopropyl Alcohol; Mafenide Acetate; Meralein Sodium; Mercufenol Chloride; Mercury, Ammoniated; Methylbenzethonium Chloride; Nitrofurazone; Nitromersol; Octenidine Hydrochloride; Oxychlorosene; Oxychlorosene Sodium; Parachlorophenol, Camphorated; Potassium Permanganate; Povidone-Iodine; Sepazonium Chloride; Silver Nitrate; Sulfadiazine, Silver; Symclosene; Thimerfonate Sodium; Thimerosal; and Troclosene Potassium.

When the cyclopropanated carbocyclic 2′-deoxynucleoside is employed for the prevention or treatment of KS, it is particularly preferred to administer it in combination with chemotherapeutics such as topoisomerase II inhibitors (e.g., etoposide), antibiotics (e.g., bleomycin), vinca alkaloids (e.g., vincristine, vinblastine), anthracyclines (e.g., doxorubicin, daunorubicin), taxanes (e.g., paclitaxol), and the like. The cyclopropanated carbocyclic 2′-deoxynucleoside can also be administered with angiogenesis inhibitors, such as thalidomide, angiostatin, endostatin, SU5416 (semaxinib), and the like, or any other suitable substance having anti-angiogenic properties. Interferon-alpha and/or retinoid (alitretinoin) can also be administered with the cyclopropanated carbocyclic 2′-deoxynucleoside in the prevention or treatment of KS.

The cyclopropanated carbocyclic 2′-deoxynucleoside can be provided to an administering physician or other health care professional in the form of a kit. The kit is a package which houses a container which contains the cyclopropanated carbocyclic 2′-deoxynucleoside in suitable form and instructions for administering the pharmaceutical composition to a subject. The kit can optionally also contain one or more additional therapeutic agents. The kit can optionally contain one or more diagnostic tools and instructions for use. For example, a kit containing a composition comprising a cyclopropanated carbocyclic 2′-deoxynucleoside in combination with one or more additional antiviral, antibacterial, and/or anti-infective agents can be provided, or separate pharmaceutical compositions containing a cyclopropanated carbocyclic 2′-deoxynucleoside and additional therapeutic agents can be provided. The kit can also contain separate doses of the cyclopropanated carbocyclic 2′-deoxynucleoside for serial or sequential administration. The kit can contain suitable delivery devices, e.g., syringes, inhalation devices, and the like, along with instructions for administrating the cyclopropanated carbocyclic 2′-deoxynucleoside and any other therapeutic agent. The kit can optionally contain instructions for storage, reconstitution (if applicable), and administration of any or all therapeutic agents included. The kits can include a plurality of containers reflecting the number of administrations to be given to a subject. In a particularly preferred embodiment, a kit for the treatment of KS is provided that includes a cyclopropanated carbocyclic 2′-deoxynucleoside and an anti-cancer agent or other therapeutic agent used to treat KS. For example, doxorubicin, bleomycin, vinblastine, vincristine, etoposide, pacilataxel, interferon alfas, recombinant interferon alfa-2a, recombinant interferon alfa-2b, and the like can be employed as additional therapeutic agents in the treatment of KS. Kits for the treatment of KSHV can also include such therapeutic agents, but preferably employ additional therapeutic agents currently employed for the treatment of viral infections such as HIV. For example, reverse transcriptase inhibitors such as zidovudine, didanosine, zalcitabine, stavudine, 3TC, and nevirapine; protease inhibitors; cytokines; immunomodulators, and anti-infectives commonly employed to combat AIDS-related infections can be employed.

The cyclopropanated carbocyclic 2′-deoxynucleosides of preferred embodiments can be administered prophylactically for the prevention of KSHV or KS. Alternatively, therapy is preferably initiated as early as possible following the onset of signs and symptoms of KS or a KSHV infection. The administration route, amount administered, and frequency of administration will vary depending on the age of the patient, condition to be treated, and severity of the condition. Contemplated amounts, dosages, and routes of administration for KSHV infections are similar to those established for the antiherpetic agent acyclovir, which is also a nucleoside analog. Detailed information relating to administration and dosages of acyclovir can be found in the Physician's Desk Reference, 47th edition, pp. 844-850, 1993 and in Hayden et al., “Antiviral Agents” in Basic Principles in the Diagnosis of Infectious Diseases, pp. 271-274). Detailed information relating to administration and dosages of therapeutic agents for treating opportunistic infections in HIV-infected individuals can be found in MMWR Morb Mortal Wkly Rep 53, RR-15, 2004. This information can be adapted in designing treatment regimes utilizing the cyclopropanated carbocyclic 2′-deoxynucleosides of preferred embodiments.

Briefly, contemplated amounts of cyclopropanated carbocyclic 2′-deoxynucleosides for oral administration to treat KSHV infections are from about 10 mg or less to about 2000 mg or more administered from about every 4 hours or less to about every 6 hours or more (or from about 4 times daily to about 6 times daily) for from about 5 days or less to about 10 days or more (40 mg/day or less to about 15,000 mg/day or more) or until there is a significant improvement in the condition. For chronic suppressive therapy for recurrent infections, or to prevent or inhibit the onset of infection in susceptible individuals, doses of from about 10 mg or less to about 1000 mg or more are orally administered once, twice, or multiple times a day, typically for up to about 12 months, or, in certain circumstances, indefinitely (from about 10 mg/day to about 1,000 mg/day). When treatment is long term, it can be desirable to vary the dosage, employing a higher dosage early in the treatment, and a lower dosage later in the treatment. For topical administration to skin lesions associated with KS, a topical preparation containing from about 10 mg or less to about 100 mg or more cyclopropanated carbocyclic 2′-deoxynucleoside per gram of preparation is typically applied in an amount sufficient to adequately cover all lesions. Higher or lower dosages can be desirable, depending upon the nature of the lesion and the patient being treated. The topical preparation is applied every three to six hours from four to six times a day for about 5 days or less to 10 days or more or until the lesions have disappeared (from about 100 mg/day or less to about 1,000 mg/day or more). The dose size per application can be adjusted depending upon the total lesion area, but preferably approximates a one cubic centimeter ribbon of preparation per sixteen square centimeters of skin surface area. For intravenous administration, from about 1 mg/kg to about 10 mg/kg is infused at a constant rate over 30 minutes or less to about 1 hour, 2 hours or more, every 6 hours or less to 8 hours or more (typically, from about 3 mg/kg/day to about 30 mg/kg/day) for about 5 days or less to about 7 days or more.

Contemplated amounts of cyclopropanated carbocyclic 2′-deoxynucleosides, methods of administration, and treatment schedules for individuals with KS are typically similar to those described above. However, longer term therapy is generally employed when treating KS than when treating a KSHV infection. For example, treatment durations of from 1 week or less up to about 1, 2, 3, 4, 5, 6, 7, 8, 9, 10, 11, 12, 16, 18, 24, 30, or 36 months or more are contemplated.

### Experiments

### Cells, Compounds and Reagents

BCBL-1, a latently KSHV-infected B cell line established from a body cavity based lymphoma, was obtained through the AIDS Research and Reference Reagent Program, Division of AIDS, NIAID, NIH (contributed by Drs. Michael McGrath and Don Ganem) (Renne, R. et al. 1996 Nat Med 2:342-6). The cells were maintained in RPMI 1640 medium supplemented with 10% fetal bovine serum (HyClone, Logan, Utah), 2 mM L-glutamine, and 1% penicillin-streptomycin-fungizone mixture (final concentrations 100 U/mL, 100 μg/mL and 0.25 μg/mL, respectively) (Cambrex, East Rutherford, N.J.) at 37° C. in 5% CO₂-containing humidified air and split at 1:10 every three to four days.

N-MCT and its southern counterpart, south-methanocarbathymidine (S-MCT), which contains the pseudosugar ring locked in the southern conformation, were synthesized as previously described (Marquez, V. E. et al. 1996 J Med Chem 39:3739-47). GCV and phorbol 12-myristate 13-acetate (PMA, also called 12-O-tetradecanoylphorbol 13-acetate, TPA) were purchased from Sigma-Aldrich (St. Louis, Mo.). CDV and 5′-ethynylthymidine (5′-ET) were provided by Dr. M. Hitchcock (Gilead Sciences, Inc. Foster City, Calif.) and Dr. M. Bobek (Roswell Park Cancer Institute, Buffalo, N.Y.), respectively. [Methyl-³H]-(N)-MCT (4.7 Ci/mmol), [5-³H]-CDV (28.0 Ci/mmol) and [8-³H]-GCV (20.4 Ci/mmol) were obtained from Moravek Biochemicals, Inc. (Brea, Calif.). N-MCT-TP, CDV-diphosphate (DP) and GCV-TP were synthesized by TriLink BioTechnologies, Inc. (San Diego, Calif.).

### BCBL-1 Culture for Evaluation of Anti-KSHV Activity

A small fraction (1-3%) of BCBL-1 cells in culture is known to spontaneously undergo lytic cycle and release KSHV virions (Kedes, D. H. et al. 1997 J Clin Invest 99:2082-6; Renne, R. et al. 1996 Nat Med 2:342-6). The antiviral effects of N-MCT and two reference anti-herpetic compounds, CDV and GCV, were determined by the relative reduction in the amounts of cytoplasmic KSHV DNA and newly released KSHV virion-associated DNA in BCBL-1 cells exposed to the test compounds, following lytic induction by phorbol ester, PMA (Renne, R. et al. 1996 Nat Med 2:342-6).

Exponentially growing BCBL-1 cells were washed three times with phosphate-buffered saline (PBS) and resuspended in serum free AIM-V w/BSA medium (Invitrogen, Carlsbad, Calif.) at 2×10⁵ cells/mL in the absence (unstimulated control) or presence of 20 ng/mL PMA. After 24 hours, unstimulated and PMA-stimulated BCBL-1 were harvested, washed once with PBS and cultured in serum free AIM-V w/BSA medium at 2×10⁵ cells/mL without PMA in the absence or presence of the test compounds at varying concentrations. After 3 days, the cells were counted by the trypan blue dye exclusion method and centrifuged at 1,500 rpm for 5 min. The supernatants were centrifuged at 3,000 rpm for 10 min before subjected to virion-derived KSHV DNA extraction and quantitation, as described below.

The cytotoxicity of the compounds was determined simultaneously in uninduced and PMA-induced BCBL-1 cells in microplates, using the XTT assay (Weislow, O, S. et al. 1989 J Natl Cancer Inst 81:577-86). In selected experiments, anti-KSHV activity of N-MCT was compared in the presence or absence of 5′-ET, a potent inhibitor of herpesvirus thymidine kinase (TK) (Nutter, L. M. et al. 1987 Antimicrob Agents Chemother 31:368-74), to investigate whether virally encoded TK played a role in the intracellular production of an active triphosphate metabolite of N-MCT, as has been demonstrated with other nucleoside analogs, such as GCV, in KSHV infected cells (Cannon, J. S. et al. 1999 J Virol 73:4786-93).

### Measurements of Cytoplasmic and Virion-Associated KSHV DNA by PCR

Low molecular weight (LMW) DNA was extracted from the pelleted cells according to Hirt's method (Hirt, B. 1967 J Mol Biol 26:365-9) and 0.1 μg of LMW DNA was used for KSHV open reading frame 65 (ORF65) PCR by a primer pair (5′-ACGGTTGTCCAATCGTTGCCTA-3′, SEQ ID NO: 2) and 5′-TCCAACTTTAAGGTGAGAGAC-3′, SEQ ID NO: 3), generating a 529 bp fragment. The ORF65 PCR reaction mixture, containing 20 mM Tris-HCl (pH 8.4), 50 mM KCl, 2.5 mM MgCl₂, 200 μM each dNTP, 0.25 U of Platinum® Taq DNA polymerase (Invitrogen), 200 μM of each primer and template DNA, was subjected to 25 cycles of PCR amplification at 94° C. for 60 sec, 60° C. for 60 sec and 72° C. for 60 sec, followed by a final extension at 72° C. for 5 min. In addition, the mitochondrial DNA primer pair (5′-TGGAGCCGGAGCACCCTATGTC-3′, SEQ ID NO: 4 and 5′-ATGGGCGGGGGTTGTATTGATG-3′, SEQ ID NO: 5) was used as an internal control for each LMW DNA PCR sample (Yang, Q. et al. 2005 J Virol 79:6122-6133). The amplified products were visualized by electrophoresis on a 1.8% agarose gel.

KSHV virions were pelleted from 300 μL of BCBL-1 culture supernatants by a microcentrifugation at 37,000 g for 2 hours at 4° C. The pelleted virions were resuspended in 150 μL PBS and treated with 20 units of DNase I (Promega, Madison, Wis.) at 37° C. for 30 min to remove cellular DNA from the samples, followed by the incubation with stop solution (20 mM EGTA) at 70° C. for 5 min. Virion-associated KSHV DNA (vDNA) was then extracted by QIAamp DNA extraction kit (QIAGEN, Valencia, Calif.) according to the manufacturer's instructions. One μL of vDNA eluted in 100 μL of elution buffer was subjected to real-time quantitative PCR using a LightCycler® instrument (Roche Applied Science, Indianapolis, Ind.). The 20-μL reaction mixture consisted of the LightCycler FastStart DNA Master SYBR Green I reagents mix (Roche Applied Science), 2.5 mM MgCl₂ and 500 nM each of KSHV ORF26 primer pair (5′-AGCCGAAAGGATTCCACCATT-3′, SEQ ID NO: 6 and 5′-TCCGTGTTGTCTACGTCCAGA-3′, SEQ ID NO: 7). Ten-fold serial dilutions of the plasmid, pKS330Bam (obtained through the AIDS Research and Reference Reagent Program, Division of AIDS, NIAID, NIH, contributed by Drs. Yuan Chang and Patrick Moore), which contained a 330 bp KSHV fragment encoding a portion of the ORF26 gene (Chang, Y. et al. 1994 Science 266:1865-9), were included in each assay as external standards to represent 10 to 107 KSHV DNA copies/tube. The number of KSHV vDNA in each supernatant sample was calculated by the LightCycler software version 3.5 (Roche Applied Science), adjusted by the cell count and expressed as copies/10⁶ cells. In selected experiments, one μL vDNA per 10⁶ cells was subjected to KSHV ORF65 PCR as described above for 30 cycles.

### Evaluation of Intracellular Phosphorylation of N-MCT

Exponentially growing BCBL-1 cells or CEM-SS cells (a human T cell line) were washed three times with PBS and cultured in serum free AIM-V w/BSA medium (Invitrogen) at 2×10⁵ cells/mL in the absence (unstimulated control) or presence of 20 ng/mL PMA. After 24 hours, unstimulated and PMA-stimulated cells were harvested, washed once with PBS and resuspended in serum free AIM-V w/BSA medium at 2×10⁵ cells/mL without PMA in the absence or presence of 10 μM N-MCT, CDV or GCV and 5 μCi/mL of the corresponding radiolabeled compound [³H]-(N)-MCT, [³H]-CDV or [³H]-GCV. Control cultures containing the same concentration of the test compounds but without the radiolabeled formulations were simultaneously set up in an identical manner to assess the cell counts and to evaluate their anti-KSHV activity. In selected experiments, 5′-ET was added at 10˜50 μM to investigate the changes in the anti-KSHV activity and intracellular phosphorylation profiles of the test compounds. The cells were harvested after 24 or 72 hours of incubation.

Upon harvest, the cells were centrifuged at 1,500 rpm for 10 min and washed once with cold PBS. The cell pellets were resuspended in 250 μL of 60% methanol and heated at 95° C. for 3 min, followed by a microcentrifugation at 12,000 g for 10 min at 4° C. The clarified supernatant fractions were evaporated under nitrogen, redissolved in 250 μL of water and subjected to HPLC separation of the phosphorylated metabolites as described in detail elsewhere (Noy, R. et al. 2002 Mol Cancer Ther 1:585-93; Zalah, L. et al. 2002 Antiviral Res 55:63-75). Fractions containing radiolabeled nucleotides were quantitated based on the known specific activity of the parent tritiated nucleoside (Noy, R. et al. 2002 Mol Cancer Ther 1:585-93; Zalah, L. et al. 2002 Antiviral Res 55:63-75). The phosphorylated metabolites of CDV were identified as CDV-phosphate, CDV-DP (active metabolite), and a phosphate ester adduct of CDV as previously described (Ho, H. T. et al. 1992 Mol Pharmacol 41:197-202).

### In Vitro DNA Synthesis Inhibition Assay

To investigate whether the triphosphate metabolite of N-MCT could directly block KSHV DNA polymerase-mediated DNA synthesis, a rapid microplate-based DNA synthesis assay (Lin, K. et al. 2000 J Virol Methods 88:219-25; Ricciardi, R. P. et al. 2004 Methods Mol Biol vol. 292) was carried out in the absence or presence of increasing concentrations of N-MCT-TP, using recombinant KSHV DNA polymerase (rPOL) and polymerase processivity factor (rPPF). KSHV rPOL and rPPF were expressed and purified from the recombinant baculovirus-vector infected Sf9 cells (Dorjsuren, D. et al. 2003 Protein Expr Purif 29:42-50). The DNA synthesis reaction was carried out in a microplate coated with a 5′-biotinylated 100-mer oligonucleotide template with a 20-mer primer annealed to its 3′-end (primed template, 0.2 pmol/well) with 10 ng each of KSHV rPOL and rPPF in a 50 μL reaction mixture, containing 50 mM (NH4)₂SO₄, 20 mM Tris-HCl (pH 7.5), 3 mM MgCl₂, 0.1 mM EDTA, 0.5 mM DTT, 2% glycerol, 40 μg/mL BSA, 0.625 μM dNTPs, and 0.125 μM digoxigenin-11-2′-deoxyuridine-5′-triphosphate (DIG-dUTP) (Roche Applied Science), at 37° C. for 60 min in the absence or presence of increasing concentrations of N-MCT-TP, CDV-DP or GCV-TP. The amounts of newly synthesized DNA, which incorporated DIG-dUTP, were determined by the DIG detection kit (Roche Applied Science) according to the manufacturer's instructions.

### Anti-KSHV Activity of N-MCT

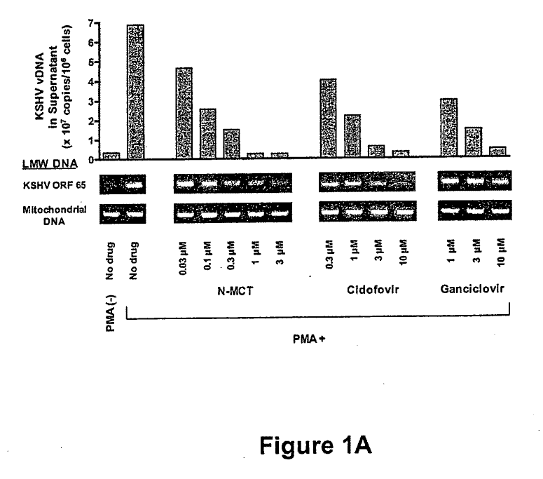

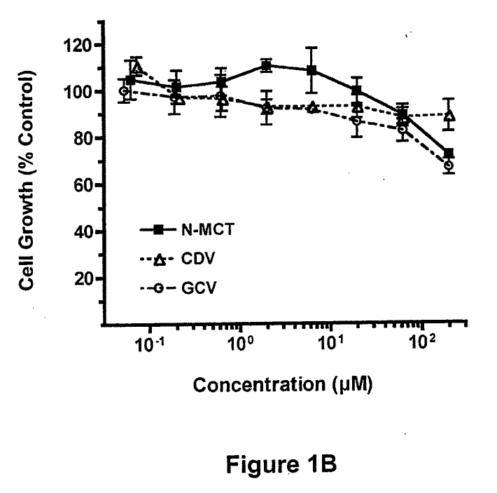

In the BCBL-1 cell-based assay developed for testing N-MCT, the number of newly released KSHV virion-associated DNA copies determined by quantitative PCR was consistently 10 to 50-fold increased (median 16.5 fold) in PMA-induced cells over uninduced control, with a corresponding increase in the amount of KSHV DNA in the cytoplasm. FIG. 1A depicts the effects of N-MCT, cidofovir (CDV) and ganciclovir (GCV) on KSHV DNA replication. The antiviral effects of the three compounds were evaluated in PMA-stimulated BCBL-1 cells, which produced over 10 to 50-fold higher levels of KSHV virions than unstimulated BCBL-1 (PMA (−), farthest left lane). After lytic replication was fully induced by PMA for 24 hours, the vigorously washed BCBL-1 cells were incubated with the test compounds at concentrations ranging from 0.03 to 10 μM. Dose-dependent decreases in KSHV virion-associated DNA (vDNA) copies as well as cytoplasmic KSHV DNA content examined in low molecular weight (LMW) DNA were demonstrated for all three compounds. The data shown are representative of 3 independent experiments.

To determine the biological effects of the test compounds specifically on lytic KSHV DNA replication, the compounds were added to the BCBL-1 culture after lytic cycle was fully induced by PMA for 24 hours. Dose-dependent decreases in KSHV vDNA and cytoplasmic KSHV DNA levels were readily observed for N-MCT, CDV and GCV at the concentrations tested from 0.03 to 10 μM (FIG. 1A) without notable cytotoxicity, although at much higher concentration (200 μM) mild cytotoxicity was detected for N-MCT and GCV in PMA-induced BCBL-1 cells. FIG. 1B depicts the cytotoxic effects of the three compounds tested in PMA-stimulated BCBL-1 cells at concentrations reaching much higher concentrations than anti-KSHV inhibitory concentrations (see Table 1).

TABLE 1

| | IC₅₀ (μM) | IC₉₀ (μM) |
|---|---|---|
| Compound | (mean ± SD) | (mean ± SD) |
| N-MCT | 0.08 ± 0.03 | 0.68 ± 0.10 |
| CDV | 0.42 ± 0.07 | 4.01 ± 2.05 |
| GCV | 0.96 ± 0.49 | 7.11 ± 0.28 |

PMA-stimulated BCBL-1 cells were cultured with increasing concentrations of N-MCT, CDV or GCV and the cell growth was determined by XTT method (Weislow, O, S. et al. 1989 J Natl Cancer Inst 81:577-86) after 72 hours and shown as % no drug control (mean±SD of triplicate wells). Modest levels of cytotoxicity were noted with N-MCT and GCV at 200 μM. The three compounds also induced similar cytotoxicity profiles in unstimulated BCBL-1 and uninfected CEM-SS cells. The experiment shown was representative of three separate assays (FIG. 1B).

N-MCT exhibited the highest anti-KSHV activity with a 50% inhibitory concentration (IC₅₀) of 0.08±0.03 μM (mean±SD) as compared to 0.42±0.07 and 0.96±0.49 for CDV and GCV, respectively (Table 1). In contrast, no antiviral activity was observed with S-MCT (data not shown), as has been reported against HSV-1 and HSV-2 (Marquez, V. E. et al. 1996 J Med Chem 39:3739-47).

### Phosphorylation of N-MCT in KSHV-Infected and Uninfected Cells

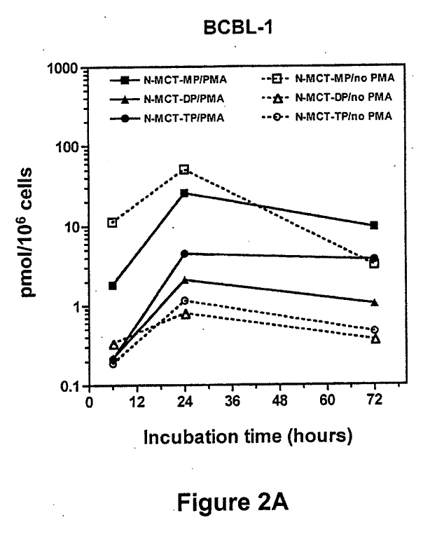

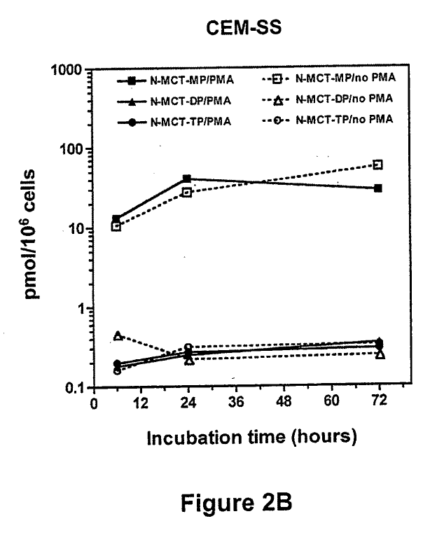

The antiviral activity of N-MCT against HSV-1 is mediated through its triphosphate metabolite produced in HSV-1-infected cells (Zalah, L. et al. 2002 Antiviral Res 55:63-75). To determine whether N-MCT inhibited lytic KSHV DNA replication through a similar mechanism, the intracellular metabolic products of N-MCT in KSHV-infected BCBL-1 cells and uninfected T lymphocyte cell line, CEM-SS cells, were investigated. The latter was used as a reference to compare the intracellular phosphorylation of N-MCT, since it is widely used to screen anti-HIV activity of various compounds, including thymidine and other nucleoside analogs (Weislow, O, S. et al. 1989 J Natl Cancer Inst 81:577-86). BCBL-1 and CEM-SS cells with or without PMA-stimulation were incubated with 10 μM N-MCT and 5 μCi/mL [³H]-(N)-MCT for 6, 24 and 72 hours, and the methanolic cell extracts were analyzed by gradient anion-exchange HPLC (Zalah, L. et al. 2002 Antiviral Res 55:63-75). The HPLC profiles clearly showed the presence of N-MCT-monophosphate (MP) in both cell lines regardless of PMA-stimulation as early as after 6 hours of incubation (FIGS. 2A and 2B). Sharp increases in N-MCT-DP and N-MCT-TP levels were also observed in BCBL-1 cells in 24 hours, especially in PMA-stimulated BCBL-1 cells, which contained 5 to 8-fold higher levels of N-MCT-DP and N-MCT-TP than unstimulated BCBL-1 (FIG. 2A). The levels of N-MCT-TP were consistently higher than N-MCT-DP in PMA-induced as well as uninduced BCBL-1 cells (FIG. 2A). In contrast to KSHV-infected BCBL-1, there were no appreciable accumulations of N-MCT-DP and N-MCT-TP in uninfected CEM-SS cells with or without PMA-stimulation (FIG. 2B). These data indicated that the intracellular phosphorylation of N-MCT to its monophosphate form could take place in both KSHV-infected and uninfected cells, but the conversion to the di- and tri-phosphorylated metabolites was significantly more efficient in KSHV-infected cells especially during lytic replication cycle.

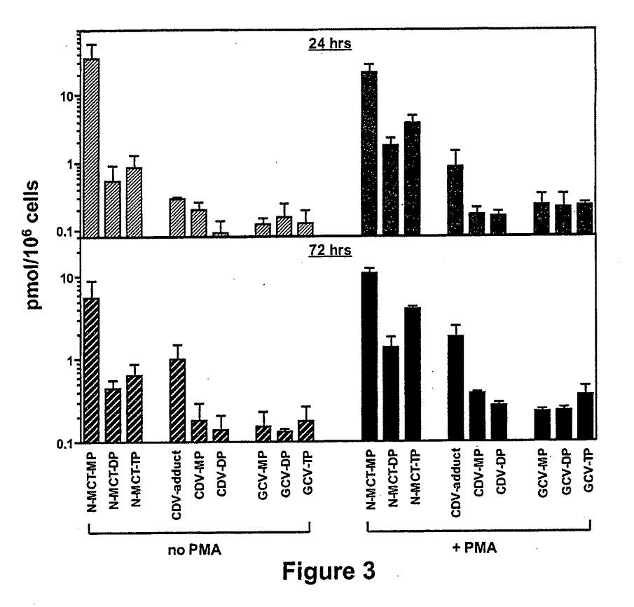

The levels of phosphorylated metabolites of N-MCT, CDV, and GCV were also compared in PMA-induced and uninduced BCBL-1 cells after 24 and 72 hours of incubation with 10 μM of each cold (unlabeled) and 5 μCi/mL of ³H-labeled compound. As shown in FIG. 3, PMA-stimulated BCBL-1 cells contained generally higher levels of phosphorylated metabolites of all three compounds as compared to unstimulated BCBL-1. The levels of N-MCT-TP were significantly higher than those of CDV-DP and GCV-TP throughout the 72 hour-incubation period especially in PMA-stimulated BCBL-1 cells.

### Herpesvirus TK Inhibitor Blocks Anti-KSHV Activity of N-MCT and N-MCT-TP Formation

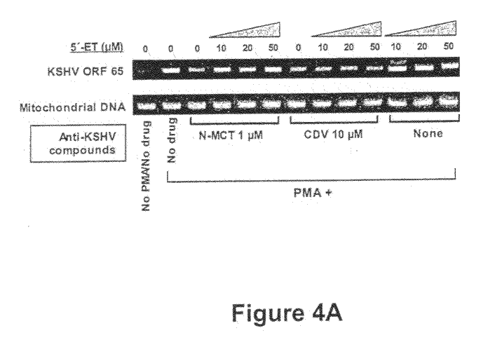

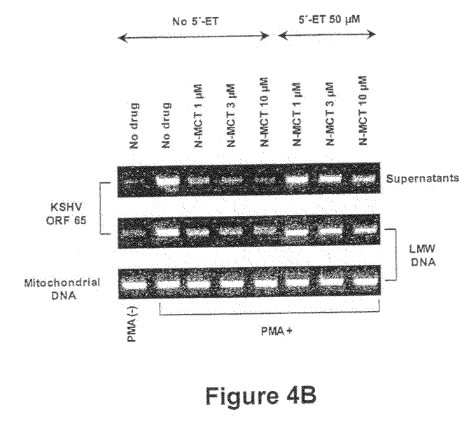

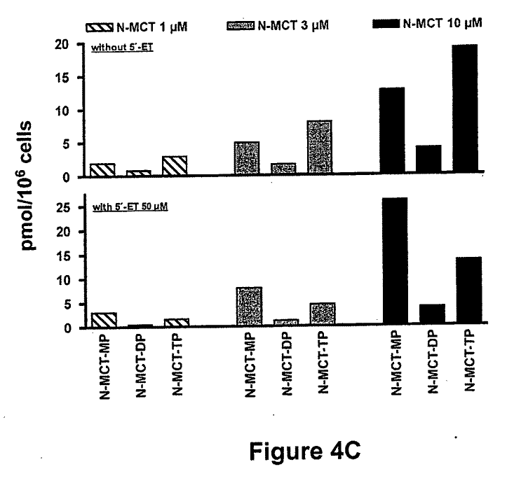

KSHV ORF21 has been reported to encode a functionally active TK (Cannon, J. S. et al. 1999 J Virol 73:4786-93; Gustafson, E. A. et al. 2000 J Virol 74:684-92). To further elucidate whether N-MCT-TP formation was directly linked to the anti-KSHV activity of N-MCT, and whether its synthesis was mediated through the virally encoded TK as has been shown in HSV-1 infected cells (Zalah, L. et al. 2002 Antiviral Res 55:63-75), the effects of 5′-ET in PMA-stimulated BCBL-1 cells treated with N-MCT were evaluated. The thymidine analog, 5′-ET, has been shown to exert a strong inhibitory activity against HSV-1 TK (Nutter, L. M. et al. 1987 Antimicrob Agents Chemother 31:368-74) as well as EBV TK (Kira, T. et al. 2000 Antimicrob Agents Chemother 44:3278-84), but not against human cellular TK (Nutter, L. M. et al. 1987 Antimicrob Agents Chemother 31:368-74). The anti-KSHV activity of N-MCT was first compared in PMA-induced BCBL-1 cells treated with N-MCT alone or in combination with varying concentrations of 5′-ET. CDV, which is converted to its active metabolite, CDV-DP, by cellular kinases (Cihlar, T. et al. 1996 Mol Pharmacol 50:1502-10; Ho, H. T. et al. 1992 Mol Pharmacol 41:197-202), was used as a reference compound. As compared to the cells treated with 1 μM N-MCT alone, marked increases in the level of cytoplasmic KSHV DNA were noted in the cells treated with a combination of 1 μM N-MCT and 10, 20 or 50 μM of 5′-ET, with the KSHV DNA level virtually returning to the baseline (no drug control) at 50 μM of 5′-ET (FIG. 4A). In contrast, the antiviral activity of CDV, tested at 10 μM to achieve a comparable inhibitory effect to 1 μM N-MCT, was not affected by the addition of 5′-ET.

The inhibitory effect of 50 μM 5′-ET on anti-KSHV activity of N-MCT was clearly demonstrated even at higher concentrations of N-MCT tested up to 10 μM. PMA-induced BCBL-1 cells were treated with 1, 3, or 10 μM N-MCT alone or in combination with 50 μM 5′-ET. The amounts of virion-associated and cytoplasmic KSHV DNA were significantly higher in the cells treated with both N-MCT and 5′-ET compared to the cells treated with N-MCT alone at all three concentrations (FIG. 4B). The intracellular levels of phosphorylated N-MCT metabolites, N-MCT-MP, N-MCT-DP and N-MCT-TP, were dose-dependently increased in the cells treated with 1, 3, or 10 μM N-MCT and 5 μCi/mL [³H]-(N)-MCT (FIG. 4C, top panel). In the presence of 50 μM 5′-ET, which significantly diminished the anti-KSHV effect of N-MCT in PMA-induced BCBL-1 cells (FIG. 4B), the levels of N-MCT-DP and N-MCT-TP in the methanolic cell extracts were substantially decreased, while there appeared to be an accumulation of N-MCT-MP (FIG. 4C, bottom). These data indicate that anti-KSHV activity of N-MCT is most likely mediated through its triphosphate metabolite, N-MCT-TP, which is converted from its precursor, N-MCT-MP, through N-MCT-DP more efficiently in KSHV-infected cells expressing the viral TK.

### N-MCT-TP Inhibits DNA Synthesis In Vitro

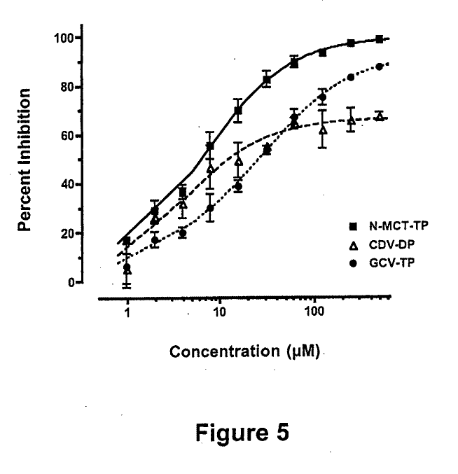

Inhibitors of KSHV POL-mediated processive DNA synthesis have been shown to be efficiently screened by a rapid microplate-based in vitro DNA synthesis assay (Ricciardi, R. P. et al. 2004 Methods Mol Biol 292:481-92). In order to further ascertain that N-MCT-TP was indeed an active metabolite of N-MCT, which blocked lytic KSHV DNA replication in cells, the inhibitory effect of N-MCT-TP on processive DNA synthesis in vitro was evaluated, using baculovirally expressed recombinant rPOL and rPPF (Dorjsuren, D. et al. 2003 Protein Expr Purif 29:42-50). The KSHV POL-specific accessory protein, KSHV PPF, which specifically associates with and holds POL onto an extending DNA template to facilitate efficient and processive DNA polymerization (Ricciardi, R. P. et al. 2004 Methods Mol Biol 292:481-92), was added to the rPOL DNA synthesis reaction mixture in order to emulate specific KSHV DNA replication. Active forms of phosphate metabolites of CDV and GCV (CDV-DP and GCV-TP, respectively) were included as a reference. All three phosphorylated compounds blocked KSHV rPOL/rPPF-mediated DNA synthesis (FIG. 5) with IC₅₀ (mean±SD) of 6.24±0.08, 14.70±2.47, and 24.59±5.60 μM for N-MCT-TP, CDV-DP and GCV-TP, respectively (Table 2).

TABLE 2

| | IC₅₀ (μM) |
|---|---|
| Compound | (mean ± SD) |
| N-MCT-TP | 6.24 ± 0.08 |
| CDV-DP | 14.70 ± 2.47 |
| GCV-TP | 24.59 ± 5.60 |

Within the concentrations tested (up to 500 μM), N-MCT-TP was the only compound that achieved greater than 90% inhibition (IC₉₀: 76.47±13.95 μM) (FIG. 5). Although CDV-DP inhibited in vitro DNA synthesis more effectively than GCV-TP at lower concentrations, its inhibitory activity appeared to level off around 60 to 70%, whereas GCV-TP dose-dependently blocked the DNA synthesis (FIG. 5).

### Results

Since the discovery of acyclovir (ACV) as a highly potent and selective anti-herpesvirus agent (Elion, G. B. et al. 1977 PNAS USA 74:5716-20), a number of nucleoside analogs have successfully been introduced to treat or prevent infections with various human herpesviruses, including HSV (α-herpesvirus), varicella-zoster virus (α-herpesvirus), and CMV (β-herpesvirus) (De Clercq, E. 2004 Nat Rev Microbiol 2:704-20). The majority of these nucleoside compounds inhibit viral replication as intracellularly activated 5′-triphosphate metabolites, which compete with natural substrates of DNA synthesis for the incorporation, resulting in the termination of viral DNA chain elongation (Ashton, W. T. et al. 1982 Biochem Biophys Res Commun 108:1716-21; Derse, D. et al. 1981 J Biol Chem 256:11447-51; Field, A. K., et al. 1983 PNAS USA 80:4139-43; Frank, K. B. et al. 1984 J Biol Chem 259:1566-9; Furman, P. A. et al. 1979 J Virol 32:72-7; Keller, P. M. et al. 1981 Biochem Pharmacol 30:3071-7; McGuirt, P. V. et al. 1984 Antimicrob Agents Chemother 25:507-9; Smith, K. O. et al. 1982 Antimicrob Agents Chemother 22:55-61). Therefore, the antiviral potency, selectivity, and cytotoxicity of the nucleoside analogs are largely dictated by their intracellular phosphorylation profiles. For example, ACV, GCV, and their oral prodrugs, valaciclovir and valganciclovir, respectively, are more efficiently mono-phosphorylated in herpesvirus-infected cells than uninfected cells, because they are better substrates for virally encoded kinases as compared to cellular nucleoside kinases (Ashton, W. T. et al. 1982 Biochem Biophys Res Commun 108:1716-2; Field, A. K. et al. 1983 PNAS USA 80:4139-43; Fyfe, J. A. et al. 1978 J Biol Chem 253:8721-7).

HSV-1 TK is a multifunctional enzyme with diverse substrate specificity, known to exhibit TK and thymidylate kinase activities (Chen, M. S. et al. 1978 J Biol Chem 253:1325-7; Chen, M. S. et al. 1979 J Virol 30:942-5). It has been shown to phosphorylate thymidine, deoxyuridine, deoxycytidine, various pyrimidine and purine analogs as well as monophosphate forms of thymidine and (E)-5-(2-bromovinyl)-2′-deoxyuridine (BVDU) (Chen, M. S. et al. 1978 J Biol Chem 253:1325-7; Chen, M. S. et al. 1979 J Virol 30:942-5; Cheng, Y. C. et al. 1983 PNAS USA 80:2767-70; Elion, G. B. et al. 1977 PNAS USA 74:5716-20; Fyfe, J. A. 1982 Mol Pharmacol 21:432-7; Keller, P. M. et al. 1981 Biochem Pharmacol 30:3071-7). The unique aspects of intracellular phosphorylation of N-MCT were first discovered in HSV-1-infected cells (Zalah, L. et al. 2002 Antiviral Res 55:63-75). The compound was found to be efficiently monophosphorylated in uninfected and HSV-1-infected cells, indicating that N-MCT was a suitable substrate for cellular TK for monophosphorylation (Zalah, L. et al. 2002 Antiviral Res 55:63-75). However, the successive conversion of N-MCT-MP to N-MCT-DP and N-MCT-TP could only be detected in the HSV-1-infected cells, and the use of an HSV-1 TK inhibitor resulted in the accumulation of N-MCT-TP in the infected cells (Zalah, L. et al. 2002 Antiviral Res 55:63-75). These data suggested that N-MCT-MP was not recognizable by cellular thymidylate kinase, and that the rate-limiting step for N-MCT activation was the conversion of N-MCT-MP to N-MCT-DP presumably catalyzed by HSV-1-encoded TK/thymidylate kinase, since N-MCT-DP was thought to be readily phosphorylated to N-MCT-TP by cytosolic nucleoside diphosphate kinase (NDK) (Zalah, L. et al. 2002 Antiviral Res 55:63-75). The discovery also suggested that N-MCT could be specifically activated (tri-phosphorylated) in cells infected with herpesviruses, if they encoded TK/thymidylate kinases capable of recognizing N-MCT-MP as an optimal substrate.

Inhibitory activities of various nucleoside analogs against KSHV replication have previously been evaluated in KSHV-infected cell lines (such as BCBL-1) lytically induced by PMA (Kedes, D. H. et al. 1997 J Clin Invest 99:2082-6; Medveczky, M. M. et al. 1997 AIDS 11:1327-32; Neyts, J. et al. 1997 Antimicrob Agents Chemother 41:2754-6). Of the compounds examined to date, CDV has been identified as one of the most potent anti-KSHV agents, while GCV was associated with only moderate levels of activity (Kedes, D. H. et al. 1997 J Clin Invest 99:2082-6; Medveczky, M. M. et al. 1997 AIDS 11:1327-32; Neyts, J. et al. 1997 Antimicrob Agents Chemother 41:2754-6). It has been surprisingly found that N-MCT blocks KSHV lytic replication in BCBL-1 cells at a 5 to 10-fold lower IC₅₀ than those of CDV and GCV without notable cytotoxicity (the 50% cytotoxic concentration of N-MCT>200 μM). As has been shown in HSV-1-infected cells exposed to N-MCT (Zalah, L. et al. 2002 Antiviral Res 55:63-75), a time and dose-dependent accumulation of N-MCT-TP almost exclusively in KSHV-infected cells was also observed, while both uninfected and infected cell lines contained abundant levels of N-MCT-MP. The data indicate that the intracellular conversion of N-MCT-MP to N-MCT-DP is most likely mediated by KSHV ORF21-encoded TK, which has been shown to exhibit thymidylate kinase activity (Gustafson, E. A. et al. 2000 J Virol 74:684-92). In the presence of a potent herpesvirus TK inhibitor, 5′-ET (Kira, T. et al. 2000 Antimicrob Agents Chemother 44:3278-84; Nutter, L. M. et al. 1987 Antimicrob Agents Chemother 31:368-74), the levels of N-MCT-DP and N-MCT-TP were significantly reduced, resulting in the abrogation of anti-KSHV activity of N-MCT. These findings further support the hypothesis that KSHV TK catalyzed phosphorylation of N-MCT-MP to N-MCT-DP, which is then intracellularly converted to N-MCT-TP by cellular NDK, and that the triphosphate form of N-MCT is directly responsible for the anti-KSHV activity. Notably, the intracellular accumulation of N-MCT-TP is significantly greater than those of CDV-DP and GCV-TP, the active metabolites of CDV and GCV, respectively, in BCBL-1 cells treated with each compound at the same concentration. While not wishing to be bound by any particular theory, it is believed that these properties may, at least in part, account for the superior anti-KSHV activity of N-MCT.

As compared to HSV-1 TK, which is known to possess a broad range of substrate specificity, KSHV TK has more restricted substrate specificity. It has been reported that KSHV TK preferentially phosphorylated thymidine derivatives, while GCV, a guanine analog, was a poor substrate for the enzyme (Gustafson, E. A. et al. 2000 J Virol 74:684-92). Although it is still possible that GCV may be phosphorylated by a KSHV ORF36-encoded phosphotransferase as has previously been suggested (Cannon, J. S. et al. 1999 J Virol 73:4786-93), it has been found that the intracellular levels of GCV-TP were, nonetheless, significantly lower than those of N-MCT-TP in KSHV-infected BCBL-1 cells, corresponding to the lower anti-KSHV efficacy of GCV as compared to N-MCT. The data further support the use of thymidine-based analogs as anti-KSHV agents. In addition to KSHV, N-MCT may also exert antiviral activity against another herpesvirus, EBV, which has been shown to encode TK with similar characteristics to KSHV TK, exhibiting thymidylate kinase activity of a substrate preference to thymidine analogs (Gustafson, E. A. et al. 1998 Antimicrob Agents Chemother 42:2923-31). Considering the lack of well-established, effective anti-EBV agents currently available, N-MCT can be an effective inhibitor against EBV replication and can be useful in treating EBV-induced malignancies.

Proportionately higher levels of N-MCT-MP than N-MCT-DP and N-MCT-TP were observed in KSHV-infected cells, whereas in acutely HSV-1-infected Vero cells the levels of N-MCT-TP were consistently higher than N-MCT-MP and N-MCT-DP (Zalah, L. et al. 2002 Antiviral Res 55:63-75). The differential phosphorylation profiles indicate that KSHV TK does not as efficiently phosphorylate N-MCT-MP as HSV-1 TK, and/or chronic KSHV infection in BCBL-1 cells employed in experiments resulted in only a modest level of viral TK expression even during lytic infection as compared to acute HSV-1 infection. Additionally, a possible difference in species-specific enzymatic activity between African green monkey and human NDKs may have played some role. Of note, the intracellular accumulation of monophosphorylated BVDU (BVDU-MP) or (E)-5-(2-iodovinyl)-2′-deoxyuridine (IVDU-MP) has directly been linked to cytostatic effects in TK-deficient tumor cells expressing HSV TK (Balzarini, J. et al. 1987 Mol Pharmacol 32:410-6). BVDU-MP and IVDU-MP were suspected to target host thymidylate synthase (TS), thereby hindering cellular DNA synthesis (Balzarini, J. et al. 1987 Mol Pharmacol 32:410-6). While both KSHV-infected and uninfected cells exposed to 10 μM N-MCT were found to contain abundant levels of N-MCT-MP, there was no significant cytotoxicity noted in either cell group until the test dose reached 200 μM. Therefore, it is unlikely that N-MCT-MP interferes with host TS in the cells exposed to the KSHV-inhibitory concentrations of N-MCT. KSHV also encodes a functional TS (Gaspar, G. et al. 2002 J Virol 76:10530-2). Although it has yet to be determined whether N-MCT-MP can interfere with virally encoded TS, the role of N-MCT-MP in KSHV inhibition is probably minimal, since the KSHV core lytic DNA replication machinery does not include KSHV TS (Russo, J. J. et al. 1996 PNAS USA 93:14862-7; Wu, F. Y. et al. 2001 J Virol 75:1487-506).

Another critical determinant of anti-herpetic activity of nucleoside-based agents is the efficiency with which the active metabolites are “misincorporated” into viral DNA. For example, S-MCT has not been associated with significant inhibitory activity against HSV-1 (Marquez, V. E. et al. 1996 J Med Chem 39:3739-47) or KSHV, despite evidence to suggest that it is an excellent substrate for virally encoded TK (Marquez, V. E. et al. 2004 J Am Chem Soc 126:543-9; Schelling, P. et al. 2004 J Biol Chem 279:32832-8). While not wishing to be bound by any particular theory, it is believed that S-MCT-TP is not a preferred substrate for DNA polymerases as compared to N-MCT-TP (Marquez, V. E. et al. 2004 J Am Chem Soc 126:543-9), illustrating the two distinct factors involved to attain antiviral activity. It has also been shown that herpesvirus polymerases possess an inherent 3′ to 5′ exonuclease activity (Marcy, A. I. et al. 1990 Nucleic Acids Res 18:1207-15; Nishiyama, Y. et al. 1983 Virology 124:221-31; Tsurumi, T. et al. 1994 J Virol 68:3354-63), as with other well-characterized DNA polymerases (Brutlag, D. et al. 1972 J Biol Chem 247:241-8; Goscin, L. P. et al. 1982 Biochemistry 21:2513-8; Huang, W. M. et al. 1972 J Biol Chem 247:3139-46). Therefore, antiviral potency of nucleoside analogs can be greatly influenced by the sensitivity vs. insensitivity (resistance) of phosphorylated metabolites to the exonuclease activity of viral polymerases. Furthermore, the processivity factors of HSV-1 and EBV polymerases, UL42 and BMRF1, respectively, have been shown to enhance the exonuclease activity of the viral polymerases, substantially reducing the extent of nucleotide misincorporation into DNA (Song, L. et al. 2004 J Biol Chem 279:18535-43; Tsurumi, T. et al. 1994 J Virol 68:3354-63). It is highly plausible that KSHV POL exhibits a similar exonuclease activity, and in the presence of KSHV PPF, the enzyme can efficiently remove mismatched nucleotides from the DNA chain during processive DNA synthesis. N-MCT-TP was shown to block in vitro DNA synthesis mediated by KSHV rPOL and rPPF more effectively than CDV-DP and GCV-TP. The data not only indicate that N-MCT-TP is efficiently incorporated into DNA, ultimately terminating the processive DNA synthesis, but also suggest that N-MCT-MP is more resistant to excision than two other reference compounds examined. In contrast to dideoxynucleoside compounds known as immediate DNA chain terminators (Atkinson, M. R. et al. 1969 Biochemistry 8:4897-904; Knopf, K. W. et al. 1981 J Virol 39:746-57; Mitsuya, H. et al. 1987 PNAS USA 84:2033-7), the active metabolites of N-MCT, CDV, and GCV do not block DNA chain elongation at the site of incorporation (Boyer, P. L. et al. 2005 J Mol Biol 345:441-50; Reid, R. et al. 1988 J Biol Chem 263:3898-904; Xiong, X. et al. 1997 Antimicrob Agents Chemother 41:594-9). Although the mechanisms are unclear, this mode of delayed chain termination may confer relative resistance to excision (Boyer, P. L. et al. 2005 J Mol Biol 345:441-50; Xiong, X. et al. 1997 Antimicrob Agents Chemother 41:594-9). The rigid conformation of the pseudosugar moiety of N-MCT may play a role in excision resistance.

Therapeutic utilities of nucleoside analogs in KS and other KSHV-induced malignancies have heretofore remained undefined. Because most KS cells are latently infected with KSHV, questions have been raised over the use of anti-herpetic compounds in the treatment of KS. However, more recent evidence suggests that KS tumorigenesis and progression require ongoing lytic replication in order to recruit new cells to infection and sustain episomal latency (Grundhoff, A. et al. 2004 J Clin Invest 113:124-36). Apart from anecdotal clinical observations, there have been a number of small reports that addressed whether anti-herpetic compounds, such as CDV or GCV, alleviated or delayed progression of KSHV-induced neoplasms in patients. While some reported tumor regression (Badiaga, S. et al. 1998 Clin Infect Dis 27:1558-9; Casper, C. et al. 2004 Blood 103:1632-4; Mazzi, R. et al. 2001 AIDS 15:2061-2; Robles, R. et al. 1999 J Acquir Immune Defic Syndr Hum Retrovirol 20:34-8), others observed no evidence of efficacy (Little, R. F. et al. 2003 J Infect Dis 187:149-53; Senanayake, S. et al. 2003 J Med Virol 71:399-403). Nevertheless, the studies describing the responders consistently found decreased levels of KSHV viral load after the treatment (Badiaga, S. et al. 1998 Clin Infect Dis 27:1558-9; Casper, C. et al. 2004 Blood 103:1632-4; Mazzi, R. et al. 2001 AIDS 15:2061-2), whereas no significant change or increases in viral load were noted in the non-responders (Little, R. F. et al. 2003 J Infect Dis 187:149-53; Senanayake, S. et al. 2003 J Med Virol 71:399-403). It is possible that the conventional anti-herpetic compounds used in the previous studies may not have effectively inhibited lytic KSHV replication in all patients. If successfully targeted, blocking lytic KSHV replication may potentially slow KS tumor progression. N-MCT can therefore benefit those with KSHV-induced malignancies.

N-MCT exhibits potent anti-KSHV activity, and is specifically triphosphorylated in KSHV-infected cells undergoing lytic replication and efficiently blocks KSHV DNA replication. The compound is suitable for use in the prevention and treatment of KSHV-induced malignancies.

Methods and compositions that are suitable for use in conjunction with aspects of the preferred embodiments are disclosed in U.S. Pat. No. 5,840,728; U.S. Pat. No. 5,629,454; and U.S. Pat. No. 5,869,666.

All references cited herein, including but not limited to published and unpublished applications, patents, and literature references are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

The term “comprising” as used herein is synonymous with “including,” “containing,” or “characterized by,” and is inclusive or open-ended and does not exclude additional, unrecited elements or method steps.

All numbers expressing quantities of ingredients, reaction conditions, and so forth used in the specification are to be understood as being modified in all instances by the term “about.” Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

What is claimed is:

1. A method of treating a Kaposi's sarcoma-associated herpes virus infection in an individual in need thereof, comprising the step of:
administering to the individual an effective Kaposi's sarcoma-associated herpes virus antiviral amount of a compound having the formula 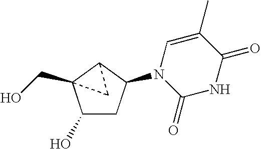
or a triphosphate thereof, in a pharmaceutically acceptable carrier.

2. The method of claim 1, wherein the effective Kaposi's sarcoma-associated herpes virus antiviral amount is from about 300 mg per day to about 15,000 mg per day.

3. The method of claim 1, wherein the compound is North-methanocarbathymidine triphosphate.

4. A pharmaceutical kit comprising:
an antiviral agent comprising a compound having the formula 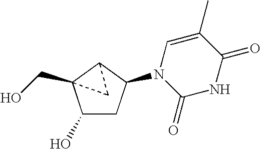
or a triphosphate thereof, in a pharmaceutically acceptable carrier; and
directions for administering the antiviral agent to a patient in need thereof for treatment of a Kaposi's sarcoma-associated herpes virus infection.

5. The pharmaceutical kit of claim 4, further comprising a reverse transcriptase inhibitor selected from the group consisting of zidovudine, didanosine, zalcitabine, stavudine, 3TC, and nevirapine, and directions for administering the reverse transcriptase inhibitor to the patient.

6. The pharmaceutical kit of claim 4, further comprising a therapeutic agent selected from the group consisting of a protease inhibitor, a cytokine, and an immunomodulator, and directions for administering the therapeutic agent to the patient.

7. The pharmaceutical kit of claim 4, wherein the compound is North-methanocarbathymidine triphosphate.

8. A method of treating a Kaposi's sarcoma in an individual in need thereof, comprising the step of:
administering to the individual an effective amount of a compound having the formula 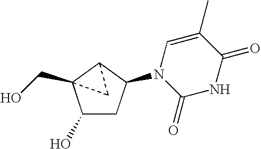
or a triphosphate thereof, in a pharmaceutically acceptable carrier.

9. The method of claim 8, wherein the effective amount is from about 40 mg per day to about 15,000 mg per day.

10. The method of claim 8, wherein the compound is North-methanocarbathymidine triphosphate.

11. A pharmaceutical kit comprising:
an anticancer agent comprising a compound having a formula 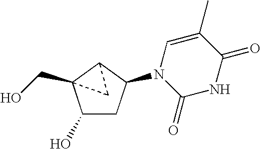
or a triphosphate thereof, in a pharmaceutically acceptable carrier; and
directions for administering the anticancer agent to a patient in need thereof for treatment of a Kaposi's sarcoma.

12. The pharmaceutical kit of claim 11, wherein the compound is North-methanocarbathymidine triphosphate

13. The pharmaceutical kit of claim 11, further comprising a chemotherapeutic agent selected from the group consisting of topoisomerase II inhibitors, antibiotics, vinca alkaloids, anthracyclines, and taxanes; and directions for administering the chemotherapeutic agent to the patient.

14. The pharmaceutical kit of claim 13, wherein the topoisomerase II inhibitor comprises etoposide.

15. The pharmaceutical kit of claim 13, wherein the antibiotic comprises bleomycin.

16. The pharmaceutical kit of claim 13, wherein the vinca alkaloid comprises vincristine or vinblastine.

17. The pharmaceutical kit of claim 13, wherein the anthracycline comprises doxorubicin or daunorubicin.

18. The pharmaceutical kit of claim 13, wherein the taxane comprises paclitaxol.

19. The pharmaceutical kit of claim 12, further comprising a therapeutic agent selected from the group consisting of an angiogenesis inhibitor, interferon-alpha, and alitretinoin, and directions for administering the therapeutic agent to the patient.

20. The pharmaceutical kit of claim 19, wherein the angiogenesis inhibitor is selected from the group consisting of thalidomide, angiostatin, semaxinib, and endostatin.


SEQUENCE LISTING (7 sequences)

SEQ ID NO: 1 - DNA, 15 bp, Artificial Sequence
  synthetic oligonucleotide
cttcattttttcttc

SEQ ID NO: 2 - DNA, 22 bp, Artificial Sequence
  synthetic primer
acggttgtccaatcgttgccta

SEQ ID NO: 3 - DNA, 21 bp, Artificial Sequence
  synthetic primer
tccaactttaaggtgagagac

SEQ ID NO: 4 - DNA, 22 bp, Artificial Sequence
  synthetic primer
tggagccggagcaccctatgtc

SEQ ID NO: 5 - DNA, 22 bp, Artificial Sequence
  synthetic primer
atgggcgggggttgtattgatg

SEQ ID NO: 6 - DNA, 21 bp, Artificial Sequence
  synthetic primer
agccgaaaggattccaccatt

SEQ ID NO: 7 - DNA, 21 bp, Artificial Sequence
  synthetic primer
tccgtgttgtctacgtccaga